(19) United States
(12) Patent Application Publication
(10) Pub. No.: US 2004/0202647 A1
(43) Pub. Date: Oct. 14, 2004

(54) Compositions and methods related to nitrotyrosine - containing compounds as antigenic agents

(75) Inventors: H. Chaim Birnboim, Ottawa (CA); John R. Webb, Manotick (CA)

Correspondence Address: BERESKIN AND PARR, SCOTIA PLAZA, 40 KING STREET WEST-SUITE 4000 BOX 401, TORONTO, ON, M5H 3Y2, CA

(73) Assignee: Novasante Inc., Ottawa (CA)

(21) Appl. No.: 10/736,555

(22) Filed: Dec. 17, 2003

Related U.S. Application Data
(60) Provisional application No. 60/433,777, filed on Dec. 17, 2002.
(60) Provisional application No. 60/460,002, filed on Apr. 4, 2003.

(51) Int. Cl. A61K 39/395; C07K 14/74; C07K 16/28

(52) U.S. Cl. 424/130.1; 530/388.1; 435/372; 530/350

(57) ABSTRACT

Herein, it is demonstrated that the posttranslational modification of the amino acid tyrosine to nitrotyrosine has profound consequences for immune recognition. Furthermore, it is demonstrated that self-proteins containing nitrotyrosine can be recognized by the immune systems as foreign and as such these same proteins can be converted to potential autoantigens. As such, this invention relates to compositions and methods for the prevention, diagnosis and treatment of conditions including autoimmune diseases, infectious diseases and cancer through the recognition of compounds containing nitrated tyrosine residues.

### CROSS REFERENCE TO RELATED APPLICATIONS

This application claims the benefit of U.S. Provisional Application No. 60/433,777, filed Dec. 17, 2002, and U.S. Provisional Application No. 60/460,002, filed Apr. 4, 2003, which are herein incorporated by reference.

### FIELD OF THE INVENTION

The invention relates to the field of nitrotyrosine and nitrotyrosine-containing compounds as antigenic agents and to related compositions, methods and uses.

### BACKGROUND OF THE INVENTION

Nitrotyrosine has been repeatedly observed in human tissues associated with inflammatory disease including, but not limited to, atherosclerosis, respiratory disease, transplant rejection, multiple sclerosis, Alzheimer's disease, inflammatory bowel disease, celiac disease, arthritis, ischemia-reperfusion injury, autoimmune diabetes, autoimmune uveitis, Helicobacter gastritis, leishmaniasis, allergic encephalomyelitis ¹. Nitrotyrosine has also been observed in a number of human tumor settings including bladder carcinoma ², colorectal carcinoma ³, breast cancer ⁴, esophageal squamous cell carcinoma ⁵, non-Hodgkin's lymphoma ⁶, lung cancer ⁷, melanoma ⁸ and experimental tumor models ^{9;10}.

The amino acid tyrosine is converted to nitrotyrosine in the presence of reactive nitrogen intermediates (RNI), effector molecules that are commonly found in inflammatory tissues as a result of upregulation of the NO-producing enzyme, inducible nitric oxide synthase (iNOS). Many of the above-mentioned inflammatory diseases, particularly the autoimmune diseases, are associated with activated autoreactive T cells and/or autoreactive antibodies. However, there is a paucity of information regarding the identity of the ‘self’ antigens recognized by these autoimmune mechanisms. Indeed, immune cells reactive with ‘self’ antigens are usually eliminated during their maturation via selective processes collectively referred to as ‘tolerance’. However, a number of recent studies have demonstrated that posttranslational protein modifications have the potential to convert self-proteins to immunogenic autoantigens ¹¹.

Autoimmune disease is the result of inappropriate recognition of ‘self’ molecules as foreign entities by the host immune system ¹². This basic tenet can be applied to a number of autoimmune diseases including, but not limited to, some forms of arthritis, juvenile diabetes, multiple sclerosis, Crohn's Disease, ulcerative colitis, systemic lupus erythematosus. In most cases, the identity of the host proteins responsible for disease onset is obscure and, in fact, during chronic infectious disease the repertoire of self-proteins recognized as foreign is thought to gradually increase in scope. This phenomenon is referred to as ‘epitope spreading’ and has been well-characterized in cases of chronic inflammation ¹². Although many different approaches are used to treat autoimmune disease, most are directed towards reducing the inflammatory process itself. These approaches, although often effective, are usually global in terms of their effects on the immune system and result in widespread immunosuppression. Thus, in many instances the patient is immunocompromised during the treatment periods and is more susceptible to infection.

Most human autoimmune diseases are characterized by periods of chronic or recurrent inflammation mediated primarily by CD4⁺ T cells secreting pro-inflammatory cytokines such as IFN-γ and TNF-α. These cytokines are capable of activating the enzyme iNOS at the site of inflammation. iNOS normally plays a role in the immune response to infection by producing nitric oxide (NO) that contributes to the destruction of invading pathogens, particularly those organisms residing within phagocytic cells of the reticuloendothelial system such as Leishmania, Mycobacteria and Salmonella. The antimicrobial activity of NO produced by iNOS is thought to stem from a number of distinct mechanisms including damage to DNA, inhibition of DNA and protein synthesis, and post-translational protein modification resulting in defective protein function. One important form of protein modification resulting from NO exposure is conversion of the amino acid residue tyrosine to nitrotyrosine ¹³. Indeed a large number of studies have documented the presence of nitrotyrosine residues in proteins both in vitro and in vivo ¹. It should also be noted that exposure of cells to NO or NO-related compounds can also result in other protein modifications in addition to the conversion of tryrosine to nitrotyrosine ¹⁰.

There is currently no permanent cure for any of the autoimmune diseases. At best, autoimmune diseases are kept under control by treatments that effectively downregulate the immune system in a global sense, usually through the use of anti-inflammatory agents. There is a strong medical need for improved detection, monitoring and treatment of autoimmune diseases. Furthermore, despite significant progress in the identification of tumor associated or tumor specific antigens, there has been limited progress in the field of tumor immunotherapy, predominantly due to the difficulties in overcoming the processes of immunological tolerance. There is a need for improved methods of immunotherapy, including the diagnosis, treatment and prevention of medical conditions such as autoimmune diseases, inflammatory diseases, cancer, and infectious diseases.

### SUMMARY OF THE INVENTION

Herein, it is demonstrated that the posttranslational modification of the amino acid tyrosine to nitrotyrosine has profound consequences for immune recognition. Furthermore, it is demonstrated that self-proteins containing nitrotyrosine can be recognized by the immune systems as foreign and as such these same proteins can be converted to potential autoantigens. Thus the present invention provides more directed and improved immunotherapies related to nitrotyrosine-containing compounds as antigenic agents.

The present inventors have identified protein-associated nitrotyrosine (which may be produced in the body as a result of chronic or recurrent inflammation) as a potent target of the immune system. Furthermore, they have demonstrated that autologous (self) proteins containing the nitrotyrosine modification can escape the processes of immunological tolerance and can function as targets of the immune system. This discovery forms the basis for novel therapeutic and prophylactic approaches for the treatment of autoimmune diseases, infectious diseases and cancer. Thus, the current invention focuses upon novel targets of the autoimmune response and also upon methods of treating autoimmune disease by interfering or otherwise disrupting the recognition of these autoantigens. This novel approach may provide a more targeted therapy for autoimmune diseases with fewer side effects than are associated with current treatment regimes. In addition, the inventors have demonstrated the ability to elicit an immune response against autologous proteins containing the nitrotyrosine moiety. Thus, this approach can also be utilized as a means to target specific cells, such as cancer cells, for attack by the immune system.

One aspect of the present invention provides novel compositions to block the interaction between self-reactive T cells or self-reactive antibodies with nitrotyrosine-containing ligands. For example, this interaction could be blocked using compounds such as, but not limited to, antibodies or antibody fragments specific for the nitrotyrosine moiety, peptides with affinity for the nitrotyrosine moiety, soluble receptors specific for nitrotyrosine, small molecule antagonists of the nitrotyrosine moiety, small molecule antagonists of T cell receptors or antibodies specific for the nitrotyrosine moiety, or compounds capable of removing or modifying the nitrotyrosine moiety. Alternatively, the interaction between the self-reactive T cells or self-reactive antibodies with nitrotyrosine-containing ligands could be blocked using compounds that block nitrotyrosine-specific T cells or nitrotyrosine-specific antibodies

In one embodiment, the invention provides a composition for treatment of autoimmune disease, said composition comprising an anti-nitrotyrosine specific monoclonal antibody or fragment thereof, together with a pharmaceutically acceptable carrier. In one embodiment, the composition is formulated for administration into a human patient.

In another embodiment, the invention provides a method for treatment of autoimmune disease using a pharmaceutical composition for administration into a human patient, said composition comprised of nitrotyrosine or nitrotyrosine-containing compounds together with a pharmaceutically acceptable carrier for the purpose of either 1) antagonizing the interaction between nitrotyrosine-specific T cells or nitrotyrosine-specific antibodies with nitrotyrosine-containing ligands or 2) inducing production of endogenous nitrotyrosine-specific T cells or nitrotyrosine specific antibodies for the same purpose.

In one embodiment, the invention provides a composition for treatment of autoimmune disease, said composition comprised of peptides or other synthetic small molecules capable of antagonizing the interaction between nitrotyrosine-specific T cells or nitrotyrosine-specific antibodies with nitrotyrosine-containing ligands, together with a pharmaceutically acceptable carrier for administration into a human patient

In one embodiment, the invention provides a composition for treatment of autoimmune disease, said composition comprised of peptides complexed with soluble forms of MHC molecules capable of antagonizing the interaction between nitrotyrosine-specific T cells with nitrotyrosine-containing ligands, together with a pharmaceutically acceptable carrier for administration into a human patient. In a further embodiment, any component of the complex is modified to contain a substance that is selectively toxic for nitrotyrosine-specific T cells when the complex interacts with such T cells.

In one embodiment, the invention provides a composition for treatment of autoimmune disease, said composition comprised of compounds capable of altering the nitrotyrosine moiety of modified self-peptides in such a manner that recognition by nitrotyrosine-specific T cells is altered, together with a pharmaceutically acceptable carrier for administration into a human patient

In one embodiment, the invention provides a composition for treatment of autoimmune disease, said composition comprised of compounds capable of inducing peripheral tolerance of nitrotyrosine-specific T cells or nitrotyrosine-specific B cells, together with a pharmaceutically acceptable carrier for administration into a human patient

In another aspect, the invention provides for various methodologies of delivering the said inhibitors to the patient. For example, in the case of antibodies against nitrotyrosine-containing ligands, these could be comprised of either humanized monoclonal antibodies specific to nitrotyrosine or autologous polyclonal antibodies generated in situ as a result of active immunization with nitrotyrosine or nitrotyrosine-containing compounds. In another embodiment, the invention provides a method of treating patients with a condition associated with nitrotyrosine containing peptides, such as an inflammatory condition, preferably an autoimmune disease, by administering the aforementioned inhibitors or antagonists to the patient.

In another aspect, the invention provides methods for screening of modulators of nitrotyrosine-containing peptides, with regard to production of such peptides and/or with respect to their activity. This can be done by administering the potential modulator to a sample comprising self-reactive T cells and nitrotyrosine-containing ligands (such as nitrotyrosine containing peptides) under conditions that are suitable for self-ractive T cell/nitrotyosine-containing ligand complex formation and monitoring the effect that the modulator has on T cell and nitrotyrosine-containing ligand complex formation. Alternatively, the potential modulator can be placed in a system that results in a known effect in the presence of nitrotyrosine-containing ligands, placing said modulator in said system that also comprises nitrotyrosine ligand and comparing the effect on the system with and without said potential modulator. In one embodiment, said system comprises cells or tissue that are affected by nitrotyrosine containing ligands in a detectable manner.

Thus, in one embodiment the invention provides a method for detecting modulators, preferably inhibitors of nitrotyrosine containing ligands and nitrotyrosine-reactive T-cells (preferably the interaction of the two components) comprising:

(a) incubating the nitrotyrosine containing ligands and nitrotyrosine-reactive T-cells under conditions that promote binding in the presence of a potential inhibitor;

(b) assaying for one or more of the following to determine the effect of the potential inhibitor on nitrotyrosine containing ligand-self reactive T-cell binding:

(i) unbound nitrotyrosine containing ligand

(ii) unbound self-reactive T-cells

(iii) unbound potential inhibitor;

(iv) bound nitrotyrosine containing ligand to self reactive T-cells;

(v) bound nitrotyrosine containing ligand to the potential inhibitor;

(vi) bound self-reactive T-cells to the potential inhibitor;

(vii) activation of T-cells bearing nitrotyrosine-specific receptors as determined by proliferation, cytokine production or other biological assays

(c) optionally comparing the assayed levels with that of a base line level or control in the absence of an inhibitor, wherein any reduction in binding of nitrotyrosine containing ligand and self reactive T-cell as compared to control or increase in unbound nitrotyrosine containing ligand or self-reactive T-cells as compared to a control would indicative of an inhibitor; or wherein any reduction of unbound nitrotyrosine containing ligand as compared to a starting level would be indicative of an inhibitor.

In another embodiment, the invention includes the inhibitor detected according to the aforementioned method.

In another aspect, the method for detecting potential inhibitors can be used for the diagnosis of an autoimmune disease or inflammatory condition wherein the nitrotyrosine containing ligand is labeled and assayed for either bound or unbound form, wherein the potential inhibitor is derived from a sample of a patient and wherein any reduction in binding of nitrotyrosine containing ligand and self reactive T-cell binding as compared to a control may be indicative of an autoimmune disease or inflammatory condition. In one embodiment of the method nitrotyrosine is present in the sample and the autoimmune disease is selected from the group of autoimmune diseases consisting of: multiple sclerosis, inflammatory bowel disease, celiac disease, arthritis, autoimmune diabetes, autoimmune uveitis, allergic encephalomyelitis, or systemic lupus erythematosus. In another embodiment of the method nitrotyrosine is produced and is indicative of a disease with an inflammatory component such as transplant rejection, Alzheimer's disease, ischemia reperfusion injury, pneumonia, or other infectious diseases.

In another embodiment, the invention provides a method for the diagnosis of an inflammatory condition comprising obtaining a sample from patient suspected of being involved in an inflammatory action, assaying the sample for nitrotyrosine containing ligand, wherein the presence of which is indicative of an inflammatory condition. In one embodiment, the level of nitrotyrosine is assayed in the sample, said level compared to a predetermined level of nitrotyrosine containing ligand indicative of an inflammatory condition to determine the inflammatory condition of the patient. In another embodiment, the samples are taken from patients with a known inflammatory disease state. In yet another embodiment the inflammatory condition is an autoimmune disease.

In another aspect, the invention provides a method for diagnosing patients related to the presence or production of nitrotyrosine containing peptides and/or the accumulation thereof in particular tissues by assaying a patient sample for the presence of nitrotyrosine containing peptides or activity. In one embodiment, the invention provides a method of diagnosing or monitoring autoimmune diseases.

In a further embodiment, the invention provides a composition for generating an immune response against nitrotyrosine-containing compounds, said composition comprising tumor-specific or tumor associated proteins or tumor-specific or tumor associated peptides that contain one (or more) tyrosine to nitrotyrosine conversions.

In another embodiment, the invention provides a composition for generating an immune response against nitrotyrosine-containing compounds, said composition comprising any self protein or peptide that contains one (or more) tyrosine to nitrotyrosine conversions.

In another embodiment, the invention provides a composition for generating an immune response against nitrotyrosine-containing compounds, said composition comprising any proteins or peptides derived from infectious disease agents that contain one (or more) tyrosine to nitrotyrosine conversions.

In another embodiment, the invention provides a methodology for overcoming tolerance to self-proteins, by immunizing with self-proteins or self-peptides that contain one (or more) tyrosine to nitrotyrosine conversions

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description. Furthermore, those embodiments of the invention described above for nitrotyrosine may apply to the other chemical modifications in amino acid residues.

### BRIEF DESCRIPTION OF THE DRAWINGS

The invention will now be described in relation to the drawings in which:

FIG. 1 is a histological section of synovial tissues from a patient with rheumatoid arthritis.

FIG. 2A is the peptide sequence for Pigeon Cytochrome C (PCC₈₈₋₁₀₄), Moth Cytochrome C (MCC₈₈₋₁₀₃) and nitrotyrosine Moth Cytochrome C (nMCC₈₈₋₁₀₃).

FIG. 2B is the chemical formulations of tyrosine and 3-nitrotyrosine.

FIG. 2C shows the IL-2 response in 2B4 cells to MCC₈₈₋₁₀₃ and nMCC₈₈₋₁₀₃C.

FIG. 3 is a graph illustrating IFNγ response in mice immunized with MCC₈₈₋₁₀₃ or nMCC₈₈₋₁₀₃ stimulated with the indicated concentrations of said peptides.

FIG. 4 are bar graphs illustrating the immune responses (Stimulation index (A,C) and IFNγ (B,D)) in PCC transgenic mice after immunization with MCC₈₈₋₁₀₃ (A,B) or nMCC₈₈₋₁₀₃ (C,D) ). FIGS. 4 E and F illustrate the IFNγ response in PCC transgenic mice (tolerant towards PCC/MCC) immunized subcutaneously with MCC₈₈₋₁₀₃ (E) or nitrated MCC₈₈₋₁₀₃ (F) peptides as per Example 4.

FIG. 5 A illustrated the CTLL-2 proliferation responses of a panel of T cell hybridomas. FIG. 5B illustrates the TCRβ chain VDJ gene usage by nMCC₈₈₋₁₀₃ specific T cell hybridoma 119-1F5.

FIG. 6 A flow cytometric analysis of MHC Class I expression (H-2D^{b} by RMA and RMA-S (TAP-deficient )cells.

FIG. 7 A flow cytometric analyses of naïve splenocytes stimulated in the presence of RMA-S cells pulsed with LCMV gp33 and nitrated LCMV gp33 peptides.

FIG. 8 A flow cytometric analyses of TCR V beta repertoire usage by naïve splenocytes stimulated in the presence of RMA-S cells pulsed with LCMV gp33 and nitrated LCMV gp33 peptides.

FIG. 9 is a graph illustrating peptide-specific cytolytic activity of T cells that are expanded after incubation of naïve splenocytes in the presence of RMA-S cells pulsed with nitrated LCMV gp33 peptides.

### DETAILED DESCRIPTION OF THE INVENTION DEFINITIONS

The following standard abbreviations for the amino acid residues are used throughout the specification: A, Ala—alanine; C, Cys—cysteine; D, Asp—aspartic acid; E, Glu—glutamic acid; F, Phe—phenylalanine; G, Gly—glycine; H, His—histidine; I, Ile—isoleucine; K, Lys—lysine; L, Leu—leucine; M, Met—methionine; N, Asn—asparagine; P, Pro—proline; Q, Gin—glutamine; R, Arg—arginine; S, Ser—serine; T, Thr—threonine; V, Val—valine; W, Trp—tryptophan; Y, Tyr—tyrosine; NY, Nitro-Tyr—3-nitrotyrosine; and p.Y., P.Tyr—phosphotyrosine.

“Adjuvant” is a substance that increases the antigenic response and is used to increase production of antibody or an immune response.

“Animal” as used herein is any animal of the animal kingdom that has a cellular immune response, such as mammals, such as humans.

“Antigen” as used herein is a substance that induces an immune response. An antigenic agent is thus a substance that induces an immune response.

“Cellular Immune Response” as used herein is a T cell response, such as a helper T (CD 4+) or cytotoxic T (CD 8+) cell response. This is as opposed to an antibody response or a non-specific immune response.

“Cell line” as used herein is a population or mixture of cells of common origin growing together after several passages in vitro. By growing together in the same medium and culture conditions, the cells of the cell line share the characteristics of generally similar growth rates, temperature, gas phase, nutritional and surface requirements. The presence of cells in the cell line expressing certain substances, for example a receptor specific for nitrotyrosine-containing compounds can be ascertained, provided a sufficient proportion, if not all, of cells in the line are present to produce a measurable quantity of the substance. An enriched cell line is one in which cells having a certain trait e.g. expression of the said receptor, are present in greater proportion after one or more subculture steps than the original cell line. Preferably the cell line is derived from one, two or three originating cells. The cell line can become more homogenous with successive passages and selection for specific traits. Clonal cells are those which are descended from a single cell. A cloned cell culture is a cell culture derived from a single cell.

“Conditions that promote” nitrotyrosine-containing ligand: T cell binding or complex formation can include a variety of conditions, including temperature, cell media (if done in vitro), the presence of other substances or carriers, such as an antigen presenting cell and/or MHC class I (in the case of cytotoxic T cell complex formation) or MHC class II (in case of helper T cell complex formation) proteins, peptides or portions thereof.

“Control” as used herein when used in the context of conducting assays can include internal or external controls familiar to those skilled in the art. It can include the establishment of base line levels in which a response or other condition can be measured by. For instance in conducting an assay for identifying modulators of nitrotyrosine-containing ligand induced cellular immune response, one can compare levels to internal controls, such as starting or base line levels of free or bound modulator or free or bound ligand or T cells. Or one can compare response levels under the same or similar conditions wherein no modulator is present or a modulator or other substance with a known affect is present.

“Epitope” is that portion of an antigen that is recognized by the immune system, such as by an antibody or T cell receptor. It itself can be an antigen.

“Hybridoma” as used herein is an immortalized cell line that produces monoclonal antibodies.

“Immortalized cell line” as used herein means a cell line that can replicate and be maintained indefinitely in in vitro cultures under conditions that promote growth, preferably at least over a period of a year or years.

“Ligand” as used herein is a substance that binds specifically, such as to a T cell receptor or antibody.

“Modulator” as used herein is a substance or set of conditions that can alter a reaction, binding, or response. For instance, a modulator of a nitrotyrosine-containing ligand induced cellular immune response is a substance that can induce or inhibit or maintain (through varying conditions) the cellular immune response.

“Nitrotyrosine-Containing Compound” as used herein is a compound that has nitrotyrosine and that can illicit an immune response and includes an antigenic self antigen that has a tyrosine to nitrotyrosine conversion.

“Self-antigen” is an autologous antigen to which an animal has “tolerance” and does not illicit an immune response.

“Self-antigen that has a tyrosine to nitrotyrosine conversion” is a self-antigen wherein as tyrosine has been changed to nitrotyrosine and as used herein are defined as those that can illicit an immune response. Said conversion can be made through post-translational modification or synthetically. A self-antigen that has a tyrosine to nitrotyrosine conversion includes modified self-protein, self-peptides and portions thereof that are antigenic epitopes comprising said conversion.

“Specific” as used herein in relation to an immune response, a cytotoxic T cell or an antibody refers to a response, cell or antibody that is directed to a particular moiety or family of moieties (compounds, antigens, or the like). For instance, a T cell line specific for a nitrotyrosine containing compound would complex with that particular compound or family of compounds, such as compound comprising an epitope thereof) as the case may be, but not to other nitrotyrosine containing compounds or to the unmodified compound (one without nitrotyrosine). Although there may be some residual, background non-specific activity, activity with the target moiety or compound would be clear and pronounced in comparison.

“T cell” as used herein includes helper T cells (CD 4+) and cytotoxic T cells (CD 8+).

Herein is provided for the first time evidence that the cellular immune system can discriminate between antigens containing conventional tyrosine versus those containing nitrotyrosine residues. Furthermore, it is shown that ‘self’ proteins containing a tyrosine to nitrotyrosine conversion can be recognized as foreign by the immune system and that recognition of nitrotyrosine likely contributes to inflammation and tissue damage during autoimmune diseases. Thus it is described herein novel compositions and methods for the treatment of inflammatory diseases, including autoimmune disease, based upon the recognition of nitrotyrosine-containing self-antigens by the immune system. Furthermore, it is shown that immunization with self proteins containing a tyrosine to nitrotyrosine conversion can induce a robust anti-self immune response. Thus it is described herein compositions and methods for the treatment of cancer and infectious disease based upon immunization with nitrotyrosine-containing compounds.

In one aspect the invention provides a method for the detection and treatment of autoimmune disease. Such diseases include but are not limited to arthritis, multiple sclerosis, diabetes, Crohn's disease, ankylosing spondylitis and others apparent to those skilled in the art.

Therapeutic interventions will target the recognition of self-proteins (not normally antigenic) that have been rendered antigenic via nitration of tyrosine residues. Disruption of recognition of these self-antigens by the host immune system represents a highly selective approach to treatment of autoimmune disease without inducing global immune suppression. Thus, the antimicrobial capacity of the immune system is expected to remain intact.

The present invention relates generally to compositions and methods for the detection, monitoring and treatment of autoimmune disease in patients. The invention is more particularly related to novel compositions and methods for blocking the interaction between T cells and/or antibodies and their cognate ligand when the ligand is comprised of an autologous protein that is modified by nitration of one or more tyrosine residues.

The present inventors have shown that RNI produced during inflammation is relatively non-specific in terms of the proteins that it targets for tyrosine modification. Thus it is entirely feasible that during inflammation, proteins from the host tissue may be subject to modification by RNI, specifically the conversion of tyrosine to nitrotyrosine. These nitrotyrosine modified ‘self-proteins’ might then be recognized as foreign by the host immune system, and the ensuing ‘anti-self’ response would contribute to a cycle of chronic inflammation.

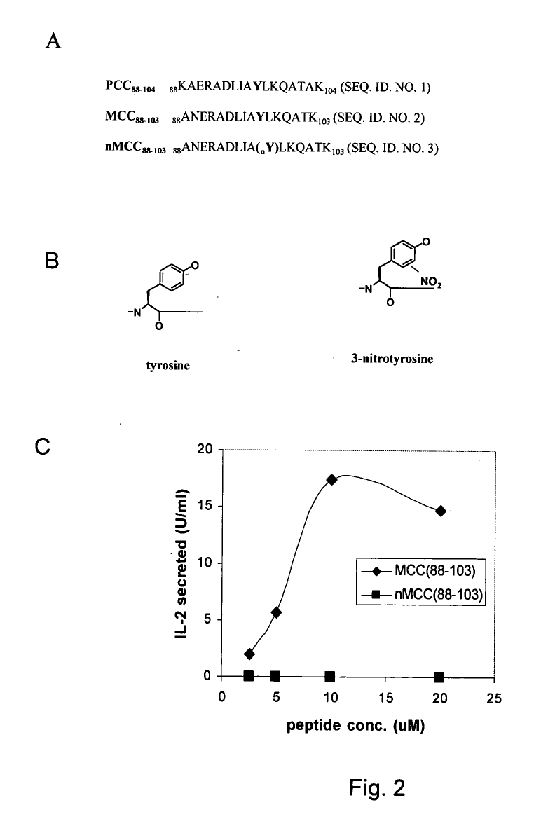

As a first step in determining whether nitrotyrosine modified ‘self-proteins’ might be a potential target for the immune system, the inventors utilized an experimental system to assess whether the immune system is capable of discriminating between proteins containing conventional tyrosine residues versus proteins containing nitrotyrosine residues. The system selected was a well-established model of antigen recognition using murine CD4⁺ T cells. The antigen-specific surface receptor (TCR) of CD4⁺ T cells recognizes specific peptides only when they are presented in the context of antigen presenting molecules called the Major Histocompatability Complex (MHC). Peptides bind to a groove in the surface of the MHC known as the “peptide binding cleft” via specific interactions between amino acids of the MHC peptide binding cleft and the peptide itself. There is loose specificity for the repertoire of peptides that can be bound to the cleft because only one or two residues of the peptide contribute to MHC binding (the so-called anchor residues). Conversely, the TCR is highly specific in terms of peptide recognition and this specificity is determined by intermolecular contacts between the TCR and both the MHC and the MHC-bound peptide. For this purpose the well-established I-E^{k}-restricted peptide from the model antigen moth/pigeon cytochrome c (MCC₈₈₋₁₀₃) was used. The MCC₈₈₋₁₀₃ peptide (FIG. 2A) comprises one of the most extensively studied model systems for understanding MHC restriction and T cell recognition and continues to be widely used as a tool for understanding the processes of T cell tolerance and T cell activation . The I-E^{k}-restricted MCC₈₈₋₁₀₃ peptide contains a single tyrosine residue (Tyr₉₇) that is not involved in MHC binding but is critically important for T cell recognition . Thus, mutated MCC₈₈₋₁₀₃ peptides containing any other amino acid in place of the Tyr₉₇ continue to bind to the I-E^{K} MHC molecule with high affinity. However, substitution of the Tyr₉₇ by any amino acid other than phenylalanine abolishes recognition by the MCC₈₈₋₁₀₃-specific T cell hybridoma 2B4, confirming that Tyr₉₇ plays a key role in recognition by the TCR. The inventors therefore tested an analogue of the MCC₈₈₋₁₀₃ peptide that contained a nitrotyrosine at position 97 rather than a tyrosine (nMCC₈₈₋₁₀₃) and found that this modification abolished recognition by the T cell hybridoma 2B4, confirming that a conversion from tyrosine to nitrotyrosine can be detected by T cells. In a converse experiment, mice expressing the I-E^{K} MHC molecule were immunized with the MCC₈₈₋₁₀₃ peptide or the nitrotyrosine-containing analogue of MCC₈₈₋₁₀₃ (nMCC₈₈₋₁₀₃). It was determined that the animals responded to immunization by activating T cells with a mutually exclusive pattern of peptide recognition. That is, animals immunized with MCC₈₈₋₁₀₃ peptide produced T cells with high specificity for MCC₈₈₋₁₀₃ peptide and weak specificity for nMCC₈₈₋₁₀₃ peptide whereas animals immunized with nMCC₈₈₋₁₀₃ peptide produced T cells with high specificity for nMCC₈₈₋₁₀₃ peptide but weak, or no, specificity for MCC₈₈₋₁₀₃ peptide. Taken together, these data provide proof of principle that the antigen recognition molecules of the immune system (in this instance TCR of CD4⁺ T cells) have the capacity to discriminate between otherwise identical peptide ligands that contain non-modified versus nitrated tyrosine residues. It was next examined whether nitration of an autologous protein might be capable of rendering it immunogenic and potentially recognizable as an autoantigen. To address this question, the inventors utilized transgenic mice that constitutively express PCC under the control of the MHC class I promoter¹⁷. These mice are unresponsive to immunization with MCC₈₈₋₁₀₃, due to the process of central tolerance whereby potentially autoreactive T cells are eliminated during their maturation in the thymus via the process of negative selection. The inventors demonstrated that when PCC transgenic mice are immunized with MCC₈₈₋₁₀₃ peptide containing a nitrotyrosine at position 97 (nMCC₈₈₋₁₀₃), PCC transgenic mice respond with a robust immune response against this modified self protein. These findings are highly relevant to the study of autoimmune diseases where chronic inflammation (and NO production) occurs. Based upon the experimental evidence regarding recognition of nitrotyrosine-containing ‘self’ peptides by CD4⁺ T cells, any ‘self’ protein modified via nitration could potentially be recognized by T cells during autoimmune disease.

In addition to CD4⁺ T cells, CD8⁺ T cells comprise another major component of the cellular immune response. CD8⁺ T cells recognize peptide antigens presented in the context of MHC class I molecules. To assess whether CD8⁺ T cells are also capable of recognizing nitrated peptides, the well-established H-2D^{b}-restricted peptide from the lymphocytic choriomeningitis virus (LCMV) glycoprotein (gp₃₃₋₄₁ or more simply gp33) ¹⁹was used. This peptide (KAVYNFATM) contains a single tyrosine (Tyr₄) that has been recently shown via crystal structure analysis to be a key TCR contact residue²⁰. As a first step in assessing the suitability of this model, the ability of nitrated and non-nitrated gp33 peptides to bind to RMA-S cells was compared. RMA-S is a mutant murine cell line that is defective for class I antigen processing and thus fails to express MHC class I molecules at the cell surface when grown at 37° C.²¹. However, peptides capable of binding to MHC class I at the cell surface can stabilize surface expressed class I as measured by flow cytometery ^{21,22}. Using this assay, nitrated and non-nitrated LCMV gp33 peptides were demonstrated to bind equally well to H-2D^{b} proving that peptides containing the amino acid analogue nitrotyrosine are fully capable of binding to MHC class I. The ability of T cells to discriminate between nitrated and non-nitrated LCMV gp33 peptides was then assessed. Splenocytes from naïve C57BI/6 mice were incubated in vitro with irradiated RMA-S cells that had been incubated with nitrated and non-nitrated LCMV gp33 peptides. After 6 days of growth, the cultures were assessed for outgrowth of CD8⁺ T cells, which was used as an indicator of T cell activation via recognition of peptide/MHC complexes through the T cell receptor. Dramatic outgrowth of CD8⁺ T cells was observed in cultures containing splenocytes plus RMA-S loaded with nitrated LCMV gp33 peptide, but not in those cultures containing splenocytes plus RMA-S loaded with non-nitrated LCMV gp33 peptide or in cultures containing splenocytes plus RMA-S only. Furthermore, analysis of the repertoire of TCR V beta chains utilized by CD8⁺ T cells growing in cultures containing splenocytes plus RMA-S loaded with nitrated LCMV gp33 peptide revealed a very restricted TCR repertoire. More specifically, of the 20 TCR V beta chains analyzed, only TCR V beta 8.3 and TCR V beta 9 were present in substantial amounts. This highly restricted TCR V beta repertoire observed in CD8⁺ T cells that are activated in the presence of RMA-S loaded with nitrated LCMV gp33 peptide but not in presence of RMA-S loaded with non-nitrated LCMV gp33 peptide is indicative of the ability of CD8⁺ T cells to discriminate between peptide ligands containing tyrosine versus those containing the inflammation-associated analogue 3-nitrotyrosine.

‘Self reactive’ T cells that could cause damage to ‘self’ tissue are normally eliminated by a process known as thymic negative selection and are thus normally absent from the peripheral tissue ¹². During thymic negative selection, T cells bind to cells of the thymus that contain ‘self’ MHC molecules plus ‘self-derived’ peptides and any positive encounter results in death of the T cell by apoptosis. Since the thymus is not an inflammatory site, it is unlikely that iNOS and iNOS-generated RNI are produced in this tissue and thus T cells would not encounter self peptides containing nitrotyrosine. The experimental data showing the existence of T cells specific for ‘self’ proteins containing nitrotyrosine residues formally demonstrates that T cells reactive with self-proteins containing nitrotyrosine escape thymic negative selection. Thus, it is highly likely that T cells capable of reacting with nitrotyrosine-containing ‘self proteins’ are present in the peripheral T cell pool and recruited to sites of inflammation in peripheral tissues. Proinflammatory cytokines produced by these self-reactive T cells after activation would propagate the inflammatory process by further activating iNOS activity resulting in further nitration of ‘self’ proteins.

In addition, it is possible that conversion of tyrosine to nitrotyrosine in the MHC molecules themselves leads to recruitment and activation of ‘self-reactive’ immune cells. Direct recognition of foreign ‘mismatched’ MHC molecules results in a robust T cell response. This process is the main contributor to rejection of transplanted tissue. As described for non-MHC nitrotyrosine-containing proteins, T cells reactive with nitrotyrosine-containing ‘self’ MHC molecules may escape thymic negative selection and may contribute to tissue damage in autoimmune disease via activation at sites of peripheral inflammation.

Thus, the current invention embodies novel compositions and methodologies for blocking the interaction of self-reactive immune cells with nitrotyrosine-containing ligands.

Furthermore, the data presented herein demonstrate that is possible to induce an immune response against nitrotyrosine-containing ‘self’ proteins through immunization. Thus, the current invention embodies novel compositions and methodologies for inducing a self-reactive immune response through immunization with nitrotyrosine-containing compounds. This finding is more specifically relevant to the setting of cancer immunotherapy in which induction of immune responses against tumor-specific or tumor-associated antigens may be beneficial in terms of inhibiting disease progression or disease prevention. Nitrotyrosine-containing ligands have been identified in a number of different tumor settings; thus, they provide an important potential target for cancer immunotherapy. The current invention further embodies novel compositions and methodologies for inducing an immune response against microbial infections through immunization with nitrotyrosine-containing compounds. Nitrotyrosine-containing ligands have been identified in a number of different infectious disease settings; thus, they provide an important potential target for immunotherapy.

### Products

Nitrotyrosine Specific T Cells and Cell Lines, Antibodies, Hybridomas.

In one embodiment, the invention provides a nitrotyrosine specific T cell line. The T cell line has a receptor for a nitrotyrosine-containing compound, such as a self-antigen comprising a tyrosine to nitrotyrosine conversion, a tumour specific protein, peptide or epitope thereof that has nitrotyrosine or tyrosine to a nitrotyrosine conversion. In one embodiment the cell line is specific to said nitrotyrosine compound. In another embodiment, the T cell does not bind or form complexes with the corresponding non-nitrotyrosine containing compound, such as a self-antigen that does not have a tyrosine to nitrotyrosine conversion or tumour specific protein, peptide or epitope that does not have the conversion. In another embodiment, the T cell line does not complex with other nitrotyrosine-containing compounds or family of compounds as the case may be. A person skilled in the art will appreciate that the specificity of the T cell line may depend on the epitope recognized by the T cell. As such, in one embodiment the T-cell line will be specific to a family of nitrotyrosine containing compounds that have the same epitope.

In one embodiment, the T cell line of the invention is a helper T (CD 4+) or cytotoxic T (CD 8+) cell line.

In another embodiment, the invention provides an isolated T cell receptor that is specific to a nitrotyrosine-containing compound, such as a self-antigen comprising a tyrosine to nitrotyrosine conversion, isolated from the T cell line of the invention. In another embodiment, the receptor is solubilized using techniques known in the art for solubilizing proteins. In another embodiment, the isolated T cell receptor comprises the amino acid SEQ. ID. NO. 4 or chemical equivalent thereof. In another embodiment, the invention provides a nucleotide sequence encoding said receptor, such as SEQ. ID. NO. 5 or degenerate sequence thereof.

A method of making a T cell line of the invention is also provided herein. In one embodiment, the method of making the T cell line that is specific to a nitrotyrosine-containing compound is made by administering the compound to an animal to induce a cellular immune response and then by subsequently isolating and culturing the T cells therefrom. Certain T cells can be selected for by assaying for their specificity to the nitrotyrosine-containing compound, for instance by binding or proliferation assays or other assays known in the art.

The T cell lines of the invention can be used in a number of applications, including in assays for identifying modulators of the nitrotyrosine-containing compound induced cellular immune responses, in diagnostic assays and in therapy by administering the T cells to a patient in need thereof, that has a condition mediated by a nitrotyrosine-containing compound and wherein the T cell is specific for said compound.

An antibody specific for a self-antigen comprising a tyrosine to nitrotyrosine conversion is also provided in the present invention. The antibody can be a polyclonal or monoclonal antibody. In one embodiment it is a monoclonal antibody. In another embodiment, the antibody is a humanized antibody. Antibodies can be humanized using techniques known in the art. IN another embodiment, the antibody is for a self-antigen that is a class 1 or class II MHC protein or fragment comprising an epitope thereof that has said conversion. In one embodiment, the invention provides a pharmaceutical composition comprising one or more of the aforementioned antibodies and a pharmaceutically acceptable carrier.

In another embodiment, the invention provides a hybridoma cell line that produces a monoclonal antibody that is specific to a self-antigen comprising a tyrosine to nitrotyrosine conversion. In another embodiment, the hybridoma is a T cell line hybridoma. Hybridoma cell lines can be made using techniques known in the art, such as by administering the self-antigen comprising the nitrotyrosine to an animal to induce an immune response. Isolating spleen cells of the animal and fusing them with an immortal cell line, such a myeloma cell line.

In another aspect of the invention, the invention provides a self-antigen that has a tyrosine to nitrotyrosine conversion and that can induce a specific immune response to said antigen to the exclusion of other antigens or family of antigens, as the case may be. In one embodiment, immune response is a cellular immune response. In another aspect, the invention provides a pharmaceutical composition comprising the self-antigen of the invention and a pharmaceutically acceptable carrier

### Applications and Methods

Diagnostic Applications and Screening Assays

The invention provides a method of screening for nitrotyrosine containing compounds or ligands and methods for diagnosing and monitoring patients with a nitrotyrosine-mediated medical condition, such as an autoimmune disease, inflammatory conditions, cancer or infectious disease. A nitrotyrosine-mediated medical condition is a condition that can be treated by targeting a nitrotyrosine-containing compound induced immune response, such as by modulating said response, such as by inducing, inhibiting or maintaining said response.

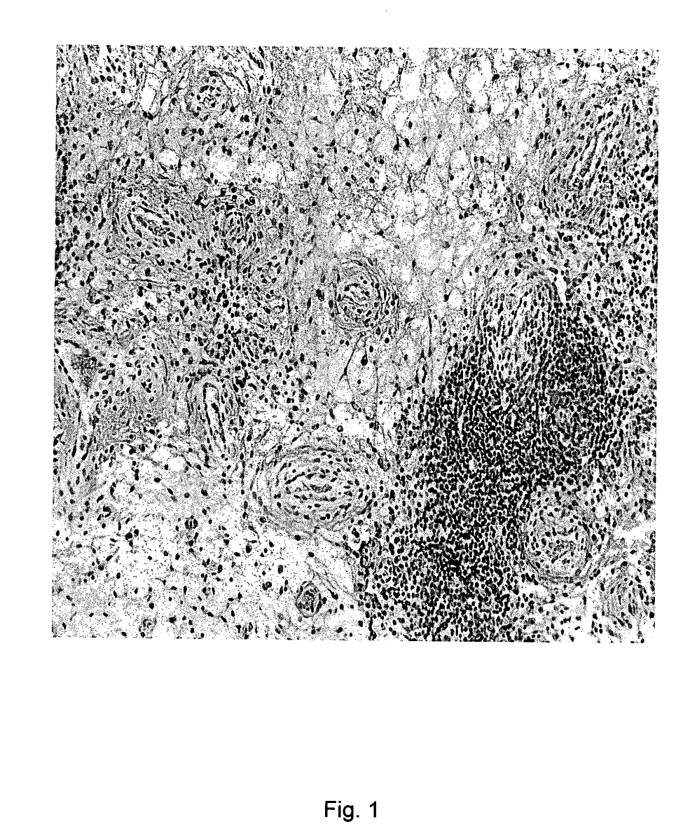

In one embodiment, such methods comprise obtaining a sample from a patient, preferably suspected to be involved in nitrotyrosine-containing compound induced immune response, such as an inflammatory or autoimmune reaction or infectious condition or cancer. Such sample can include tissue or cell samples, blood samples, stool or urine samples. The sample can then be screened for nitotyrosine-containing ligands using techniques known in the art, such as antibody staining, as seen in FIG. 1, radiolabeling or calorimetric assays using antibodies or other molecules that bind to nitrotyrosine-containing ligands. The presence of such nitrotyrosine-containing ligands or elevated levels thereof as compared to samples with known normal states would indicate the presence of inflammatory states or resurgence of an autoimmune condition or other nitrotyrosine-related condition.

In another embodiment, a diagnostic method of the invention can comprise the identification and isolation of T cells specific to nitrotyrosine modified self-antigens. The presence of such cells or elevated levels thereof as compared to samples with known normal states would indicate the presence of inflammatory states or resurgence of an autoimmune condition or another nitrotyrosine-related condition, such as cancer.

As such, in one embodiment, the invention provides a method of inducing a cellular immune response specific to a nitrotyrosine containing compound comprising administering an effective amount of the nitrotyrosine containing compound to an animal or in vivo or in vitro animal model, such as a cell or tissue culture. In one embodiment, the animal is a human. An effective amount as used in this context is an amount that can illicit the desired response. This method can be used in the treatment of a condition wherein a cellular immune response is warranted, for instance the administration of a tumour specific antigen comprising nitrotyrosine to a patient having said tumour. Alternatively, it can be used to immunize patients against a condition, such as an autoimmune disease.

In another embodiment the nitrotyrosine-containing compound is a self-antigen having a tyrosine to nitrotyrosine conversion. In yet another embodiment, the nitrotyrosine-containing compound is a tumour specific protein, peptide or fragment thereof having nitrotyrosine or a tyrosine to nitrotyrosine conversion and that is capable of inducing a cellular immune response. In one embodiment T cells specific to the nitrotyrosine-containing compound, such as helper T cells or cytotoxic T cells are generated. These in turn can in one embodiment be isolated and cultured.

In another embodiment, the invention provides a method of inhibiting a cellular immune response specific to a nitrotyrosine-containing compound comprising administering an effective amount of an inhibitor of said cellular immune reponse, such as an anti-nitrotyrosine-containing compound antibody. In one embodiment, the nitrotyrosine-containing compound is a self-antigen having a tyrosine to nitrotyrosine conversion.

In one embodiment, the invention provides a method of identifying a modulator of a nitrotyrosine-containing compound, such as a ligand induced cellular immune response comprising: incubating an effective amount of nitrotyrosine-containing ligand and T-cells specific to said nitrotyrosine-containing ligand under conditions that promote ligand/T cell complex formation in the presence of a potential modulator. Conditions that promote complex formation can include, temperature, media, antigen presenting cells, class I (for cytotoxic T cells) or II (for helper T cells) MHC proteins, peptides or functional fragments thereof to promote ligand/t cell complex formation.

A person skilled in the art would appreciate that one can first incubate the ligand and T cell(s) under conditions that promote complex formation and then add the potential modulator and monitor the affects. Alternatively one can incubate the ligand and potential modulator or the T cell and the potential modulator under conditions for instance that promote complex formation or ligand/T cell complex formation and then add the T cell or ligand as the case may be and monitor the affects on ligand/T cell complex formation, preferably under conditions that promote such complex formation. In one embodiment, one can then assay for one or more of the following to determine the effect of the potential modulator on nitrotyrosine-containing ligand: T-cell complex formation: unbound nitrotyrosine-containing ligand; unbound T-cells; unbound potential modulator; bound nitrotyrosine-containing ligand to T-cells; bound nitrotyrosine-containing ligand to the potential modulator; bound T-cells to the potential modulator; activation of T-cells bearing nitrotyrosine-specific receptors as determined by proliferation and/or cytokine production and/or expression of ligand specific T cell receptors. The levels assayed can be compared to a control wherein any change from a control level is indicative that the potential modulator is a modulator.

In one embodiment, the nitrotyrosine-containing ligand is a self-antigen having a tyrosine to nitrotyrosine conversion or a tumour specific antigen, protein or peptide.

Wherein the modulator is an inhibitor of the immune response any reduction in binding of nitrotyrosine-containing ligand and T-cell as compared to control or higher levels of unbound nitrotyrosine-containing ligand or T-cells as compared to a control or lower levels of free modulator or higher levels of bound modulator and ligand or modulator and T cell; or lower level of activated T cells as compared to a control would be indicative of an inhibitor. For instance, the inhibitor may be an antibody against the nitrotyrosine-containing compound, ligand, self-antigen, tumour specific nitrotyrosine-containing antigen or the nitrotyrosine-containing compound specific T cell receptor of the T cell. In another embodiment, wherein the modulator is an inducer of the immune response wherein any increase in binding of nitrotyrosine-containing ligand and T-cell as compared to control or lower levels of unbound nitrotyrosine-containing ligand or T-cells as compared to a control or higher levels of free modulator or lower levels of bound modulator and ligand or modulator and T cell; or higher level of activated T cells as compared to a control would be indicative of an inducer

In another embodiment, the invention provides a method of identifying self-antigens having a tyrosine to nitrotyrosine conversion that are antigenic comprising: administering the converted self-antigen to an animal and detecting CD8+ and/or antibody production or antigen/cell or antigen/antibody complex formation, wherein said production or complex formation is indicative of an antigenic converted self-antigen. In another embodiment, the assay can be done in an in vitro model.

In one embodiment, the invention provides a method of identifying a medical condition that is associated with or mediated by a nitrotyrosine-containing compound by:

(a) obtaining a sample from the patient that is affected by said condition;

(b) screening the sample for one or more of the following:

(i) nitrotyrosine-containing compounds;

(ii) nitrotyrosine-containing compounds in complex with T cells; or

(iii) nitrotyrosine-containing compound specific T cells;

(c) identifying said nitrotyrosine-containing compound;

(d) comparing the results of b and c with a control sample obtained from condition free tissue,

wherein the presence of nitrotyrosine containing compounds or specific T cells or complex formation is indicative of a medical condition, associated with a nitrotyrosine containing compound. In another embodiment, the medical condition can be identified by administering a modulator of nitrotyrosine-containing compound induced immune response to a patient having or suspected of having a related condition or to tissue sample of said patient and monitoring the affects on the disease state. In another embodiment, nitrotyrosine is present in the sample and the condition is an autoimmune disease selected from the group of autoimmune diseases consisting of: multiple sclerosis, inflammatory bowel disease, celiac disease, arthritis, autoimmune diabetes, autoimmune uveitis, allergic encephalomyelitis, or systemic lupus erythematosus. In another embodiment, nitrotyrosine is present in the sample and is indicative of a disease with an inflammatory component such as transplant rejection, Alzheimer's disease, ischemia reperfusion injury, pneumonia, leishmaniasis. In another embodiment, administration of nitrotyrosine containing compound, such as self antigen or tumour specific antigen, protein or peptide that comprises nitrotyrosine or where a tyrosine has been converted to nitrotyrosine, induces cell lysis of a disease affected tissue and is indicative of a nitrotyrosine-containing compound-mediated condition.

### Therapeutic Applications (Methods and Compositions)

In a preferred embodiment, an immune response specific for a cancerous cell can be generated by immunizing with a tumor-specific or tumor-associated peptide or tumor-specific or tumor-associated protein containing a nitrotyrosine for tyrosine substitution. This can augment, or induce a tumour specific immune response.

In another preferred embodiment, an immune response specific for an infected cell can be generated by immunizing with a host or infectious agent-derived peptide or host or infectious agent-derived protein containing a nitrotyrosine for tyrosine substitution.

In another preferred embodiment, the immune response that occurs during autoimmune disease can be disrupted using a therapeutic substance that modulates or detects nitrotyrosine-containing ligands or T cells specific for said ligands. In one embodiment substance(s) that can modulate or detect nitrotyrosine containing ligands are antibodies to said ligands or T cells, or more specifically T cell receptors for said ligands. One skilled in the art can readily prepare the antibodies using techniques known in the art such as those described by Kohler and Milstein, Nature 256, 495 (1975) and in U.S. Pat. Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference). Within the context of the present invention, antibodies are understood to include monoclonal antibodies, polyclonal antibodies, antibody fragments (e.g., Fab, and F(ab′)2) and recombinantly produced binding partners.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region that recognizes a nitrotyrosine containing peptide or ligand of the invention (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Monoclonal or chimeric antibodies specifically reactive with a protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982); and PCT Publication WO 92/06193 or EP 0239400). Humanized antibodies can also be commercially produced (Scotgen Limited, 2 Holly Road, Twickenham, Middlesex, Great Britain.)

Specific antibodies, or antibody fragments reactive against a protein of the invention may also be generated by screening expression libraries encoding immunoglobulin genes, or portions thereof, expressed in bacteria with peptides produced from nucleic acid molecules of the present invention. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)).

The antibodies may be labeled with a detectable marker including various enzymes, fluorescent materials, luminescent materials and radioactive materials. Examples of suitable enzymes include horseradish peroxidase, alkaline phosphatase, β-galactosidase, or acetylcholinesterase; examples of suitable fluorescent materials include umbelliferone, fluorescein, rhodamine, dichlorotriazinylamine fluorescein, dansyl chloride or phycoerythrin; an example of a luminescent material includes luminol; and examples of suitable radioactive material include S-35, Cu-64, Ga-67, Zr-89, Ru-97, Tc-99m, Rh-105, Pd-109, In-111, I-123, I-125, I-131, Re-186, Au-198, Au-199, Pb-203, At-211, Pb-212, Y-90 and Bi-212. The antibodies may also be labeled or conjugated to one partner of a ligand binding pair. Representative examples include avidin-biotin and riboflavin-riboflavin binding protein. Methods for conjugating or labeling the antibodies discussed above with the representative labels set forth above may be readily accomplished using conventional techniques.

Antibodies reactive against nitrotyrosine-containing compounds or T cell receptors or other compounds that can form complexes with said compounds or receptors(e.g., enzyme conjugates or labeled derivatives) may be used to detect a nitrotyrosine-containing compound (e.g. peptide, protein, ligand, self antigen), or specific receptor for said compounds of the invention in various samples. For example, they may be used in any known immunoassays that rely on the binding interaction between an antigenic determinant of a compound or protein of the invention and the antibodies. Examples of such assays are radioimmunoassays, western immunoblotting, enzyme immunoassays (e.g., ELISA), immunofluorescence, immunoprecipitation, latex agglutination, hemagglutination, and histochemical tests. Thus, the antibodies may be used to identify or quantify the amount of a compound or protein of the invention in a sample.

A sample may be tested for the presence or absence of a nitrotyrosine containing compounds, peptides or proteins by contacting the sample with an antibody specific for an epitope of a nitrotyrosine-containing compound, peptide or protein which antibody is capable of being detected after it becomes bound to a nitrotyrosine containing compound, peptide or protein in the sample, and assaying for antibody bound to a nitrotyrosine containing compound, peptide or protein in the sample, or unreacted antibody.

In a method of the invention, a predetermined amount of a sample or concentrated sample is mixed with antibody or labeled antibody. The amount of antibody used in the method is dependent upon the labeling agent chosen. The resulting protein bound to antibody or labeled antibody may be isolated by conventional isolation techniques, for example, salting out, chromatography, electrophoresis, gel filtration, fractionation, absorption, polyacrylamide gel electrophoresis, agglutination, or combinations thereof.

The sample or antibody may be insolubilized, for example, the sample or antibody can be reacted using known methods with a suitable carrier. Examples of suitable carriers are Sepharose or agarose beads. When an insolubilized sample or antibody is used protein bound to antibody or unreacted antibody is isolated by washing. For example, when the sample is blotted onto a nitrocellulose membrane, the antibody bound to a protein of the invention is separated from the unreacted antibody by washing with a buffer, for example, phosphate buffered saline (PBS) with bovine serum albumin (BSA).

When labeled antibody is used, the presence of a nitrotyrosine containing peptide can be determined by measuring the amount of labeled antibody bound to a protein of the invention in the sample or of the unreacted labeled antibody. The appropriate method of measuring the labeled material is dependent upon the labeling agent.

When unlabelled antibody is used in a method of the invention, the presence of a nitrotyrosine containing peptide can be determined by measuring the amount of antibody bound to the nitrotyrosine containing peptide using substances that interact specifically with the antibody to cause agglutination or precipitation. In particular, labeled antibody against an antibody specific for a protein of compound of the invention, can be added to the reaction mixture. The antibody against an antibody specific for a protein or compound of the invention can be prepared and labeled by conventional procedures known in the art which have been described herein. The antibody against an antibody specific for a compound or protein of the invention may be a species specific anti-immunoglobulin antibody or monoclonal antibody, for example, goat anti-rabbit antibody may be used to detect rabbit antibody specific for a protein or compound of the invention.

In another embodiment, the antibodies inhibit nitrotyrosine containing ligand-self reactive T cell interaction.

In another embodiment, peptide modeling can be used to develop a self-reactive T-cell receptor mimetic that can bind nitrotyrosine containing ligands to inhibit binding to the self-reactive T-cell receptor.

In another embodiment, compounds can be screened for their ability to inhibit or antagonize nitrotyrosine containing ligand/nitrotyrosine-reactive T-cell receptor interactions by assays such as administering the potential inhibtor with nitrotyrosine containing ligand/nitrotyrosine-reactive T-cell receptor and monitor binding using known techniques in the art, such as radiolabeling or colourmetric assays, where at least one component is labeled to enable detection and monitoring of binding activity. One could also compare binding activity in the presence and absence of the potential inhibitor. For example, the inhibitory capacity of a compound(s) could be measured by bioassays that measure proliferation, activation or cytokine production by nitrotyrosine reactive T cells or could be biochemical in nature such as standard receptor/ligand binding assays.

The present invention encompasses within its scope the compounds determined to inhibit the interaction of nitrotyrosine containing ligand/nitrotyrosine-reactive T-cell receptor, and uses thereof.

In another embodiment, the invention provides a composition for treatment of autoimmune disease or other inflammatory conditions; said composition comprising an anti-nitrotyrosine specific monoclonal antibody together with a pharmaceutically acceptable carrier. Preferably, the composition is a parenteral composition and can be administered to a human patient accordingly. In one embodiment, the invention provides a method for treating a medical condition associated with a nitrotyrosine-containing compound induced cellular immune response comprising administering to a patient in need thereof a modulator of said immune response. In one embodiment, the nitrotyrosine-containing compound is a self-antigen. In another embodiment the medical condition is an inflammatory or autoimmune disease. In another embodiment, modulator is an inhibitor of the nitrotyrosine-induced cellular immune response, for example, an antibody to said nitrotyrosine-containing compound or T cell specific receptor to said compound. In one embodiment the medical condition is cancer and the self-antigen a tumor-associated or tumor-specific peptide or protein containing a tyrosine to nitrotyrosine conversion together with a pharmaceutically acceptable carrier which induces a specific immune response to said tumor. In one embodiment the medical condition is an infectious disease caused by a cell or microbe infection and the modulator is a cellular or microbial peptide or a cellular or microbial protein containing a tyrosine to nitrotyrosine conversion that induces an antibody and/or cellular immune response against cells that are infected with the microbe. In one embodiment the medical condition is an autoimmune disease that is associated with a self-antigen comprising a tyrosine to nitrotyrosine conversion and the modulator is an anti-nitrotyrosine self-antigen specific monoclonal antibody. In one embodiment, the invention provides a method of preventing a medical condition associated with a nitrotyrosine-containing compound comprising immunizing a patient with said nitrotyrosine containing compound

In another embodiment, the invention provides a method for treatment of autoimmune disease or other inflammatory conditions using a pharmaceutical composition for administration into a human patient; said composition comprised of nitrotyrosine or nitrotyrosine-containing compounds together with a pharmaceutically acceptable carrier for the purpose of either 1) antagonizing the interaction between nitrotyrosine-specific T cells with nitrotyrosine-containing ligands or 2) inducing production of endogenous anti-nitrotyrosine specific antibodies for the same purpose.

In another embodiment, the invention provides a composition for treatment of autoimmune disease or other inflammatory conditions; said composition comprised of peptides or other synthetic small molecules capable of antagonizing the interaction between nitrotyrosine-specific T cells with nitrotyrosine-containing ligands (such as the antibodies to nitrotyrosine containing ligands mentioned above or inhibitors identified using the screening methods noted above), together with a pharmaceutically acceptable carrier for administration into a human patient

In another embodiment, the invention provides a composition for treatment of autoimmune disease or other inflammatory conditions; said composition comprised of compounds capable of altering the nitrotyrosine moiety of modified self-peptides in such a manner that recognition by nitrotyrosine-specific T cells is altered, together with a pharmaceutically acceptable carrier for administration into a human patient

In yet another embodiment, the invention provides a composition for treatment of autoimmune disease or other inflammatory conditions; said composition comprised of compounds capable of inducing peripheral tolerance of nitrotyrosine-specific T cells, together with a pharmaceutically acceptable carrier for administration into a human patient

In another aspect, the invention provides a method for treatment of autoimmune disease or other inflammatory conditions; said method involving delivery of compounds capable of inducing peripheral tolerance of nitrotyrosine-specific T cells, together with a pharmaceutically acceptable carrier for administration into a human patient

### Pharmaceutical Compositions

The above described substances that are used to induce a response against a compound containing a tyrosine to nitrotyrosine conversion or to modulate nitrotyrosine containing compounds or production thereof or compounds that are determined to be able to inhibit or antagonize nitrotyrosine containing ligands and self-reactive T-cell receptors may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals.

Administration of a therapeutically active amount or an effective amount of pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of the substance to elicit a desired response in the individual. Dosage regimes may be adjusted to provide the optimum therapeutic response. For example, several divided doses may be administered daily or the dose may be proportionally reduced as indicated by the exigencies of the therapeutic situation.

An active substance may be administered in a convenient manner such as by injection (subcutaneous, intravenous, etc.), oral administration inhalation, transdermal, parenteral application, or rectal administration. Depending on the route of administration, the active substance may be coated in a material to protect the compound from the action of enzymes, acids and other natural conditions that may inactivate the compound. If the active substance is a nucleic acid encoding, for example, a peptide that modulates the activity of a nitrotryosine containing peptide it may be delivered using techniques known in the art.

The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456. As will also be appreciated by those skilled, administration of substances described herein may be by an inactive viral carrier.

The following non-limiting examples are illustrative of the present invention.

### EXAMPLES

### Example 1

### Expression of Nitrotvrosine in a Patient with Rheumatoid Arthritis

Methods:

A histological section of synovial tissue was taken surgically from a patient with rheumatoid arthritis at the time of joint replacement. Tissues were fixed in formalin and embedded in paraffin wax. Sections (4 μm thick) were taken and processed for immunohistochemistry. After removal of wax with toluene, samples were rehydrated. Nonspecific IgG binding was blocked with swine serum and sections were incubated with 0.5 μg/ml of a commercially available rabbit polyclonal anti-nitrotyrosine antibody raised against nitrated KLH (Upstate Biotechnology) for 16 hours. Sections were then incubated with a peroxidase conjugated goat anti-rabbit immunoglobulins Immunolabelling was detected using diaminobenzidine and then lightly counterstained with hematoxylin.

Results:

The result of the anti-nitrotyrosine immunohistological stain is shown in FIG. 1, where the dark deposit indicates the location of nitrotyrosine in the tissue section. Staining of the extracellular matrix is particularly prominent but specific cell types are also occasionally stained. This illustrates that nitrotryosine peptides are prominent in tissues involved in a prototypic autoimmune disease, rheumatoid arthritis.

### Example 2

### The Ability of T Cells to Discriminate Between Tyrosine and Nitrotyrosine-Containing Compounds

Methods:

Chinese Hamster Ovary cells transfected with the murine MHC class II molecule I-E^{K} (CHO(I-E^{K})) and the MCC₈₈₋₁₀₃-specific, I-E^{K}-restricted T hybridoma (2B4) were provided by Dr. Mark Davis (Stanford University). CHO(I-E^{K}) and 2B4 cells were incubated in the presence or absence of the indicated amounts of the synthetic peptide MCC₈₈₋₁₀₃ or the same peptide containing a nitrotyrosine in place of a tyrosine at position 97 (nMCC₈₈₋₁₀₃) for 24 hours at 37° C. in standard tissue culture medium (RPMI 1640 with 10% FCS, 25 mM HEPES, 50 uM 2-ME). After 24 hr of incubation the culture supernatants were removed and the amount of IL-2 secreted into the medium by the 2B4 cells was measured by standard bioassay using the IL-2-dependent cell line CTLL-2²³.

Results:

FIG. 2C is a graph illustrating the concentration of IL-2 present in the supernatant of 2B4 cells grown in the presence of normal peptide (MCC₈₈₋₁₀₃) and nitrotyrosine containing peptide nMCC₈₈₋₁₀₃. IL-2 production by 2B4 is an indication of T cell receptor/ligand binding and subsequent T cell activation. As previously described¹⁸, 2B4 responded to MCC₈₈₋₁₀₃ peptide presented in the context of I-E^{k} by synthesizing IL-2 in a dose-dependent manner. In contrast, 2B4 was completely non-responsive to stimulation with a synthetic peptide (nMCC₈₈₋₁₀₃) containing a nitrotyrosine in place of Tyr₉₇, even at the highest concentrations tested. This observation provided clear evidence that modification of tyrosine to nitrotyrosine impacts on the process of T cell recognition.

### Example 3

### The Ability of T Cells To Discriminate Between Tyrosine and Nitrotyrosine-Containing Ligands After in Vivo Immunization

Methods:

CBA (H2^{k}) mice were immunized with either MCC₈₈₋₁₀₃ or nMCC₈₈₋₁₀₃ peptides and their immune response were assessed using in vitro recall assays. Mice were immunized subcutaneously with the indicated synthetic peptide plus incomplete Freund's adjuvant (IFA) following standard protocols. Seven days post-immunizationin the mice were euthanized and single cell suspensions were prepared from draining (popliteal) lymph nodes. Cells (2×10⁵/well) were incubated in 96 well plates in the presence and absence of the indicated concentrations of the synthetic peptide MCC₈₈₋₁₀₃ or the same peptide containing a nitrotyrosine in place of a tyrosine at position 97 (nMCC₈₈₋₁₀₃) at 37° C. in standard tissue culture medium (RPMI 1640 with 10% FCS, 25 mM HEPES, 50 uM 2-ME). After 3 days of incubation the culture supernatants were recovered and assayed for the presence of secreted IFN-γ by standard cytokine capture ELISA using commercially available reagents (BD Biosciences)

Results:

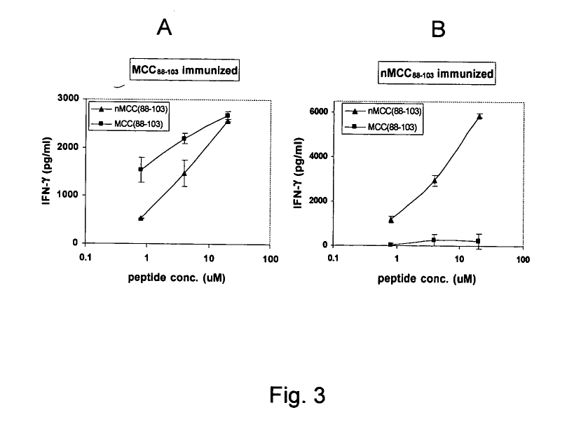

Draining lymph node cells from mice immunized with MCC₈₈₋₁₀₃ peptide secreted IFN-γ in a dose dependent manner in response to in vitro stimulation with MCC₈₈₋₁₀₃ peptide (FIG. 3). These cells also responded weakly to stimulation with nMCC₈₈₋₁₀₃ peptide. The response to the latter was, however, significantly weaker than the response to MCC₈₈₋₁₀₃ peptide. Strikingly, mice immunized with nMCC₈₈₋₁₀₃ peptide had the opposite pattern of recognition. Cells from nMCC₈₈₋₁₀₃-immunized animals secreted IFN-γ in response to in vitro stimulation with nMCC₈₈₋₁₀₃ peptide but were completely unresponsive to MCC₈₈₋₁₀₃ peptide. The presence of a robust nMCC₈₈₋₁₀₃-specific immune response provided strong evidence that conversion of the single tyrosine residue of MCC₈₈₋₁₀₃ to nitrotyrosine does not significantly impact on the ability of this peptide to be presented by I-E^{k}. This is consistent with previous results showing that Tyr₉₇ does not make direct contact with the peptide binding groove of I-E^{k}18;20. As IFN-γ is used as an indicator of T cell receptor/ligand binding and subsequent T cell activation, the results show the ability of T cells to discriminate between tyrosine and nitrotyrosine-containing peptides after in vivo immunization.

### Example 4

### Immunization with an Autologous Peptide Containing a Tyrosine to Nitrotyrosine Conversion Overcomes the Process of Central Tolerance and Provokes a Robust Anti-Self Immune Response

Methods:

Transgenic mice that constitutively express PCC under the control of the MHC class I promoter ¹⁷⁹ were immunized with either MCC₈₈₋₁₀₃ or nMCC₈₈₋₁₀₃ peptides and their immune response were assessed using in vitro recall assays. Mice were immunized subcutaneously with the indicated synthetic peptide plus incomplete Freund's adjuvant (IFA) following standard protocols. Seven days post-immunizationin the mice were euthanized and single cell suspensions were prepared from draining (popliteal) lymph nodes. Cells (2×10⁵/well) were incubated in 96 well plates in the presence and absence of the indicated concentrations of the synthetic peptide MCC₈₈₋₁₀₃ or the same peptide containing a nitrotyrosine in place of a tyrosine at position 97 (nMCC₈₈₋₁₀₃) at 37° C. in standard tissue culture medium (RPMI 1640 with 10% FCS, 25 mM HEPES, 50 uM 2-ME). After 3 days of incubation 100 ul of culture supernatant was recovered and assayed for the presence of secreted IFN-γ by standard cytokine capture ELISA using commercially available reagents (BD Biosciences). The remaining cells were pulsed with [³H}-thymidine (0.5 uCi/well) for an additional 8 hrs and the level of [³H}-thymidine incorporation was determined and used an indicator of cellular proliferation.

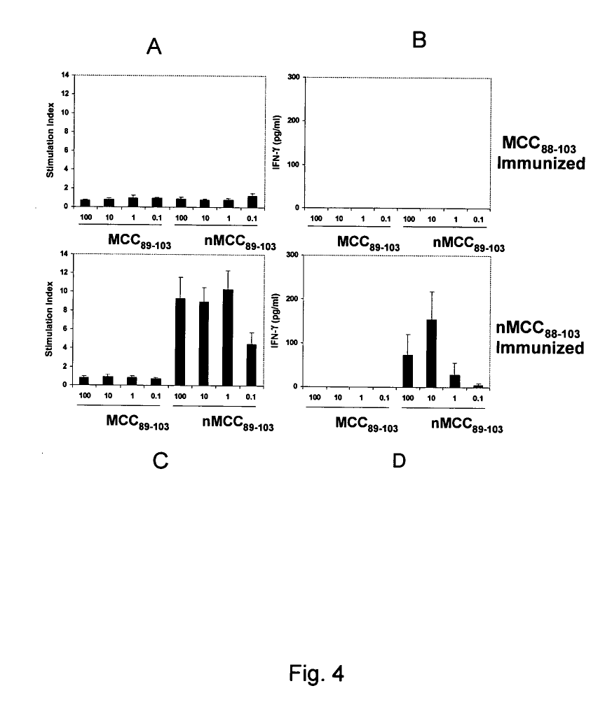

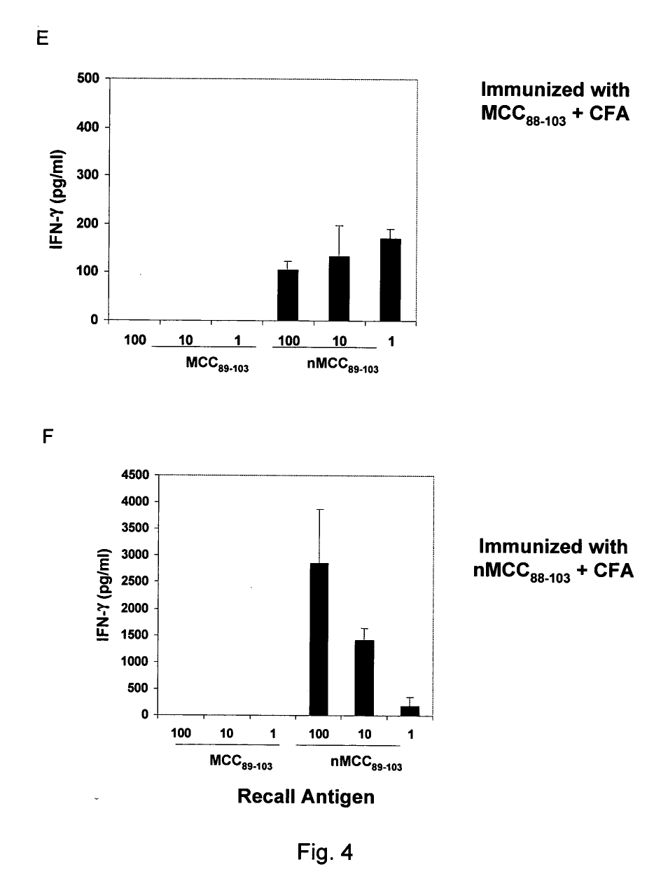

In a second experiment (results depicted in FIGS. 4 E and F) the IFNγ response was measured in PCC transgenic mice (tolerant towards PCC/MCC) immunized subcutaneously with MCC₈₈₋₁₀₃ (E) or nitrated MCC₈₈₋₁₀₃ (F) peptides. Ten days post immunization draining lymph node cells were recovered and stimulated in vitro with varying amounts of MCC₈₈₋₁₀₃ or nitrated MCC₈₈₋₁₀₃ peptide. Responsiveness was measured in terms of IFN-γ released into the supernatant in response to antigen as measured by capture ELISA.

Results

To determine whether nitration of an autologous protein might be capable of rendering it immunogenic and potentially recognizable as an autoantigen transgenic mice that constitutively express PCC under the control of the MHC class I promoter were used ¹⁹. These mice are unresponsive to immunization with MCC₈₈₋₁₀₃, due to the process of central tolerance whereby potentially autoreactive T cells are eliminated during their maturation in the thymus via the process of negative selection. In agreement with earlier findings ¹⁷, there was no detectable MCC₈₈₋₁₀₃-specific cellular immune response in PCC transgenic mice after immunization with MCC₈₈₋₁₀₃ peptide (FIG. 4). However, immunization of PCC transgenic mice with nMCC₈₈₋₁₀₃ peptide elicited a robust cellular immune response against nMCC₈₈₋₁₀₃, as measured by antigen-specific in vitro proliferation and cytokine (IFN-γ) production.

### Example 5

### T Cell Hybridomas Raised Against the Nitrotyrosine-Containing Ligand nMCC₈₈₋₁₀₃ are Highly Responsive to nMCC₈₈₋₁₀₃ but not MCC₈₈₋₁₀₃

Methods:

Splenocytes from wild type (CBA) and PCC-transgenic mice that had been immunized with nMCC₈₈₋₁₀₃ peptide were stimulated in vitro for 10 days with nMCC₈₈₋₁₀₃ peptide and then fused with BW5147 cells to generate T cell hybridomas. A panel of 12 T cell hybridomas specific for nMCC₈₈₋₁₀₃ was generated (3 from wild type CBA mice and 9 from PCC transgenic mice). Chinese Hamster Ovary cells transfected with the murine MHC class II molecule I-E^{K} (CHO(I-E^{K})) and T hybridomas were incubated in the presence or absence of the indicated synthetic peptide MCC₈₈₋₁₀₃ or the same peptide containing a nitrotyrosine in place of a tyrosine at position 97 (nMCC₈₈₋₁₀₃) for 24 hours at 37° C. in standard tissue culture medium (RPMI 1640 with 10% FCS, 25 mM HEPES, 50 uM 2-ME). After 24 hr of incubation the culture supernatants were removed and the amount of IL-2 secreted into the medium by the 2B4 cells was measured by standard bioassay using the IL-2-dependent cell line CTLL-2²³.

Results:

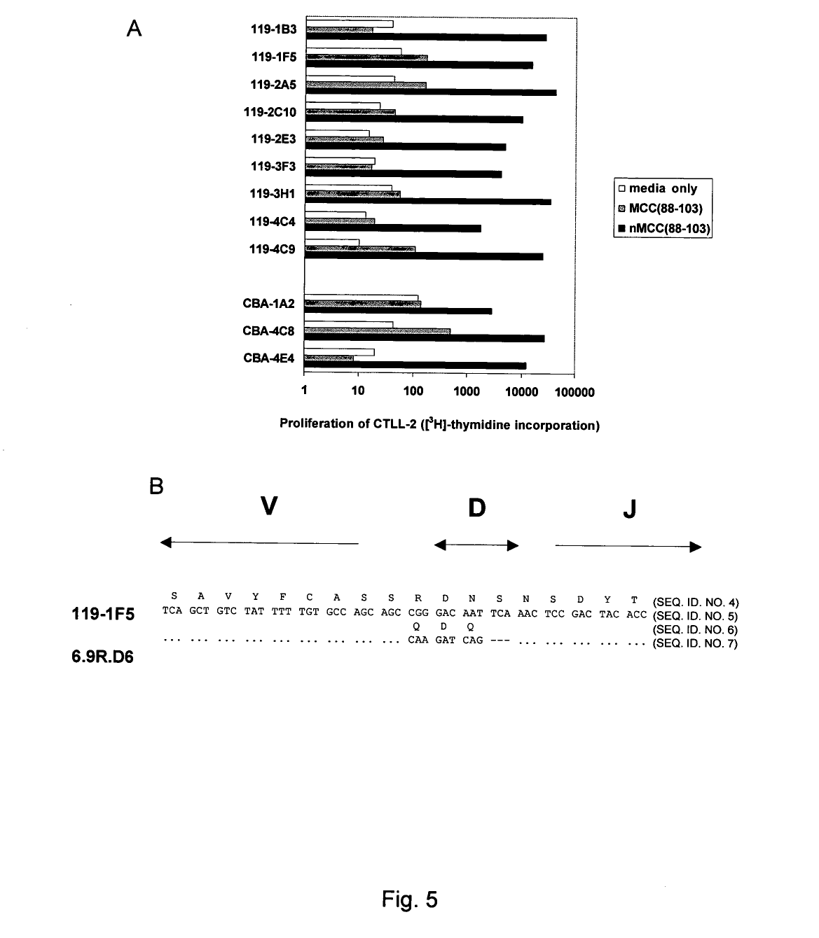

Although the T cell hybridomas varied in terms of the absolute amount of IL-2 produced in response to antigen stimulation, all 12 hybridomas showed exquisite sensitivity and specificity for nMCC₈₈₋₁₀₃ compared to MCC₈₈₋₁₀₃ peptide; only 2 of the hybridomas (119-4C9 and CBA-4C8) exhibited weak responsiveness to non-modified MCC₈₈₋₁₀₃ peptide (FIG. 5A). TCR β chain usage was evaluated for one of the hybridomas derived from PCC transgenic mice (119-1F5) by RT-PCR using degenerate primers specific for the TCR β chain gene. The sequence of the TCR β chain of 119-1F5 was very similar to that of the MCC₈₈₋₁₀₃-specific T cell clone 6.9R.D6 ²⁴ (see FIG. 5B). Both clones use the combination of V_{β}1 and J_{β}1.2; however, they differ in the area of the D region. Specifically, the TCR β chain of the nMCC₈₈₋₁₀₃-reactive T cell hybridoma contains a positively charged arginine residue within the D region whereas the β chain of the MCC₈₈₋₁₀₃-reactive clone 6.9R.D6 does not. The presence of a positively charged residue in this position of the TCR might be expected based upon the critical role of the Tyr₉₇ for T cell recognition and the addition of a negative charge upon conversion of tyrosine to nitrotyrosine. In addition, a recent description of the crystal structure of MCC₈₈₋₁₀₃ bound to I-E^{k} indicates that the aromatic ring of Tyr₉₇ is in close contact with the neighboring residue K₉₉, and that Tyr₉₇ plays a role in proper positioning of K₉₉ which is a critical TCR contact residue¹⁶. By introducing a negative charge on Tyr₉₇, it is conceivable that the interaction between Tyr₉₇ and K₉₉ has been disrupted. Regardless of the mechanism, it is interesting to note that the very subtle alteration of TCR β chain usage found on nMCC₈₈₋₁₀₃-specific T cells is sufficient to allow their escape from negative selection in PCC transgenic mice. Once again, this finding provides unequivocal evidence that nitrated peptides are unlikely to participate in the process of thymic negative selection and that T cells bearing TCRs capable of recognizing nitrated self-peptides escape this process and enter the periphery.

### Example 6

### CD8⁺ T Cells with a Distinct TCR V Beta Repertoire are Preferentially Activated when Splenocytes from Naïve C57BI/6 Mice are Cultured In Vitro with RMA-S Cells Pulsed with Nitrated LCMV gp33 Peptide

Methods:

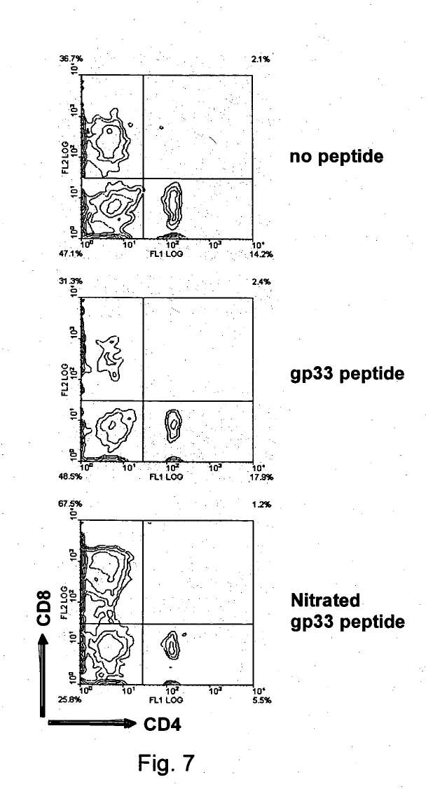

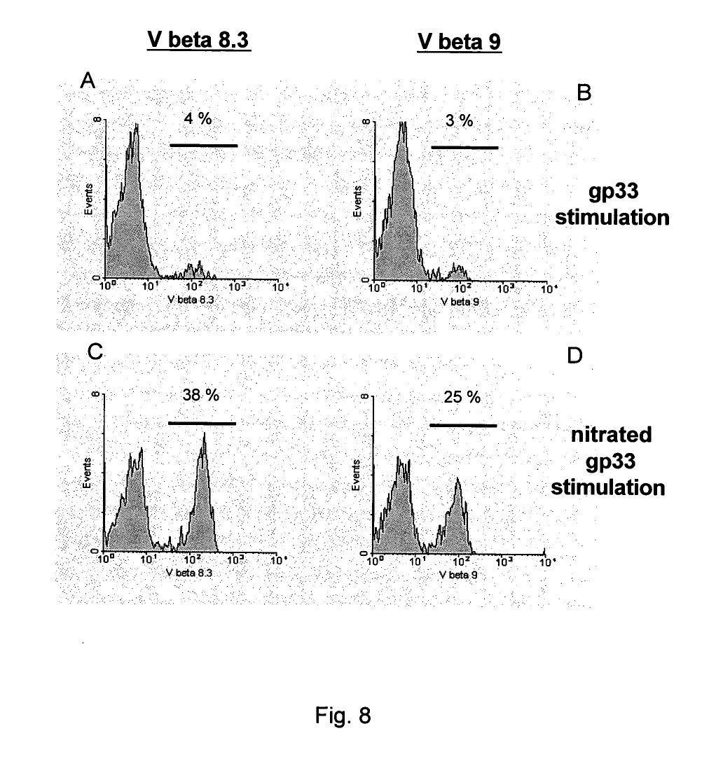

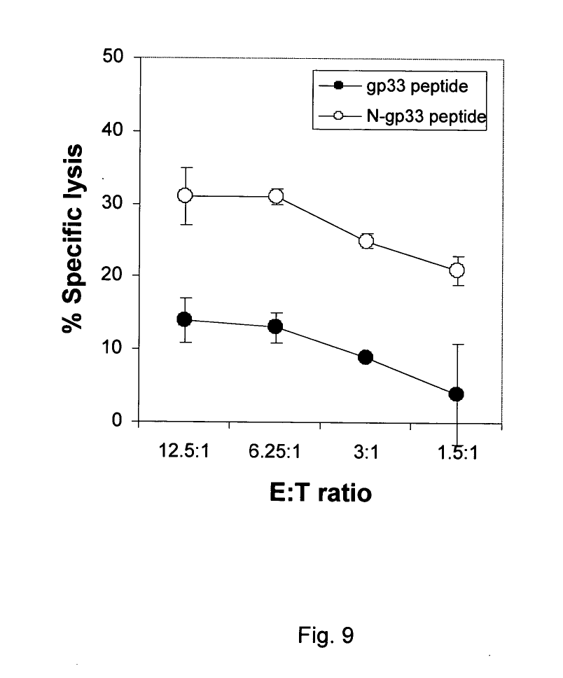

Splenocytes from naïve, wild type C57BI/6 mice (1×10⁷) were incubated in vitro with RMA-S tumor cells (1×10⁶) that had been pulsed, or not, with specific peptide (gp33-41 peptide from LCMV or gp33-41 peptide from LCMV that contained a nitrotyrosine in place of a tyrosine). After 4 days of culture IL-2 was added (10 U/mi) and culture was continued for another 2 to 5 days. Blasts cells arising in the culture were analyzed as described for CD4 CD8 phenotype (FIG. 7), TCR V beta gene usage (FIG. 8) and specific cytolytic activity (FIG. 9).

Results:

A dramatic and rapid outgrowth of CD8⁺ T cells with a highly restricted TCR V beta gene usage is observed when splenocytes from naïve C57BI/6 mice are incubated in vitro with RMA-S tumor cells pulsed with gp33-41 peptidecontaining a nitrotyrosine in place of a tyrosine. This outgrowth is not observed when splenocytes are incubated in vitro with RMA-S tumor cells pulsed with conventional gp33-41 peptide or with RMA-S tumor cells not pulsed with peptide. CD8⁺ T cells undergoing expansion after exposure to nitrated gp33 peptide also demonstrate specific lysis of EL4 tumor cells pulsed with nitrated gp33 peptide versus EL4 tumor cells pulsed with non-nitrated gp33 peptide.

While the present invention has been described with reference to what are presently considered to be the preferred examples, it is to be understood that the invention is not limited to the disclosed examples. On the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### Full Citations for References Referred to in the Specifications

1. Ischiropoulos, H. 1998. Biological tyrosine nitration: a pathophysiological function of nitric oxide and reactive oxygen species. Arch.Biochem.Biophys. 356:1-11.

2. Ehsan, A., F. Sommer, A. Schmidt, T. Klotz, J. Koslowski, S. Niggemann, G. Jacobs, U. Engelmann, K. Addicks, and W. Bloch. 2002. Nitric oxide pathways in human bladder carcinoma. The distribution of nitric oxide synthases, soluble guanylyl cyclase, cyclic guanosine monophosphate, and nitrotyrosine. Cancer 95:2293-2301.

3. Kondo, S., S. Toyokuni, T. Tsuruyama, M. Ozeki, T. Tachibana, M. Echizenya, H. Hiai, H. Onodera, and M. Imamura. 2002. Peroxynitrite-mediated stress is associated with proliferation of human metastatic colorectal carcinoma in the liver. Cancer Left. 179:87-93.

4. Samoszu, M., M. L. Brennan, V. To, L. Leonor, L. Zheng, X. Fu, and S. L. Hazen. 2002. Association between nitrotyrosine levels and microvascular density in human breast cancer. Breast Cancer Res. Treat. 74:271-278.

5. Kato, H., T. Miyazaki, M. Yoshikawa, M. Nakajima, Y. Fukai, N. Masuda, H. Ojima, K. Tsukada, Y. Nishida, and H. Kuwano. 2001. Expression of nitrotyrosine is associated with angiogenesis in esophageal squamous cell carcinoma. Anticancer Res. 21:3323-3329.

6. Mendes, R. V., A. R. Martins, G. de Nucci, F. Murad, and F. A. Soares. 2001. Expression of nitric oxide synthase isoforms and nitrotyrosine immunoreactivity by B-cell non-Hodgkin's lymphomas and multiple myeloma. Histopathology 39:172-178.

7. Pignatelli, B., C. Q. Li, P. Boffetta, Q. Chen, W. Ahrens, F. Nyberg, A. Mukeria, I. Bruske-Hohlfeld, C. Fortes, V. Constantinescu, H. Ischiropoulos, and H. Ohshima. 2001. Nitrated and oxidized plasma proteins in smokers and lung cancer patients. Cancer Res. 61:778-784.

8. Ekmekcioglu, S., J. Ellerhorst, C. M. Smid, V. G. Prieto, M. Munsell, A. C. Buzaid, and E. A. Grimm. 2000. Inducible nitric oxide synthase and nitrotyrosine in human metastatic melanoma tumors correlate with poor survival. Clin. Cancer Res. 6:4768-4775.

9. Sandhu, J. K., H. F. Privora, G. Wenckebach, and H. C. Birnboim. 2000. Neutrophils, nitric oxide synthase, and mutations in the mutatect murine tumor model. Am.J.Pathol. 156:509-518.

10. Haqqani, A. S., J. F. Kelly, and H. C. Birnboim. 2002. Selective nitration of histone tyrosine residues in vivo in mutatect tumors. J.Biol.Chem. 277:3614-3621.

11. Doyle, H. A. and M. J. Mamula. 2002. Posttranslational protein modifications: new flavors in the menu of autoantigens. Curr.Opin.Rheumatol. 14:244-249.

12. Ermann, J. and C. G. Fathman. 2001. Autoimmune diseases: genes, bugs and failed regulation. Nat.Immunol. 2:759-761.

13. Bogdan, C. 2001. Nitric oxide and the immune response. Nat.Immunol. 2:907-916.

14. Parmiani, G., C. Castelli, P. Dalerba, R. Mortarini, L. Rivoltini, F. M. Marincola, and A. Anichini. 2002. Cancer immunotherapy with peptide-based vaccines: what have we achieved? Where are we going? J.Natl.Cancer Inst. 94:805-818.

15. Yu, Z. and N. P. Restifo. 2002. Cancer vaccines: progress reveals new complexities. J.Clin.Invest 110:289-294.

16. Fremont, D. H., S. Dai, H. Chiang, F. Crawford, P. Marrack, and J. Kappler. 2002. Structural basis of cytochrome c presentation by IE(k). J.Exp.Med. 195:1043-1052.

17. Oehen, S., L. Feng, Y. Xia, C. D. Surh, and S. M. Hedrick. 1996. Antigen compartmentation and T helper cell tolerance induction. J.Exp.Med. 183:2617-2626.

18. Reay, P. A., R. M. Kantor, and M. M. Davis. 1994. Use of global amino acid replacements to define the requirements for MHC binding and T cell recognition of moth cytochrome c (93-103). J.Immunol. 152:3946-3957.

19. Nguyen, L. T., M. F. Bachmann, and P. S. Ohashi. 2002. Contribution of LCMV transgenic models to understanding T lymphocyte development, activation, tolerance, and autoimmunity. Curr. Top.Microbiol.Immunol. 263:119-143.

20. Achour, A., J. Michaelsson, R. A. Harris, J. Odeberg, P. Grufman, J. K. Sandberg, V. Levitsky, K. Karre, T. Sandalova, and G.Schneider. 2002. A structural basis for LCMV immune evasion: subversion of H-2D(b) and H-2K(b) presentation of gp33 revealed by comparative crystal structure.Analyses. Immunity. 17:757-768.

21. Ljunggren, H. G., N. J. Stam, C. Ohlen, J. J. Neefjes, P. Hoglund, M. T. Heemels, J. Bastin, T. N. Schumacher, A. Townsend, K. Karre, and 1990. Empty MHC class I molecules come out in the cold. Nature 346:476-480.

22. De Bruijn, M. L., T. N. Schumacher, J. D. Nieland, H. L. Ploegh, W. M. Kast, and C. J. Melief. 1991. Peptide loading of empty major histocompatibility complex molecules on RMA-S cells allows the induction of primary cytotoxic T lymphocyte responses. Eur.J.Immunol. 21:2963-2970.

23. Gillis, S., M. M. Ferm, W. Ou, and K. A. Smith. 1978. T cell growth factor: parameters of production and a quantitative microassay for activity. J.Immunol. 120:2027-2032.

24. McElligott, D. L., S. B. Sorger, L. A. Matis, and S. M. Hedrick. 1988. Two distinct mechanisms account for the immune response (Ir) gene control of the T cell response to pigeon cytochrome c. J.Immunol. 140:4123-4131.

25. Boon, Thierry and Van den Eynde, Benoit, 2003. Tumour Immunology: Editorial Overview. Curr. Opinion in Immunology 15:129-130

### Figure Legends

FIG. 1 shows as histological section of synovial tissue from a patient with rheumatoid arthritis. Tissue sections were stained with rabbit anti-nitrotyrosine polyclonal antiserum (Upstate Biotechnology) followed by goat anti-rabbit antibody conjugated to horseradish peroxidase plus substrate. The dark deposit indicates the location of nitrotyrosine in the tissue section. Staining of the extracellular matrix is particularly prominent but specific cell types are also occasionally stained.

FIG. 2.A Alignment of PCC₈₈₋₁₀₃, MCC₈₈₋₁₀₃ and nMCC₈₈₋₁₀₃ peptides showing the position of the single tyrosine residue (Y₉₇) which is converted to nitrotyrosine in nMCC₈₈₋₁₀₃. B. Schematic comparison of the side chains of tyrosine and nitrotyrosine. C. Response of the hybridoma 2B4 ²⁰ to peptides MCC₈₈₋₁₀₃ and nMCC₈₈₋₁₀₃. 5×10⁴ 2B4 T cell hybridoma cells were mixed with 5×10⁴ I-E^{k}-transfected CHO cells plus the indicated concentration of MCC₈₈₋₁₀₃ or nMCC₈₈₋₁₀₃ peptides. After 24 h supernatants were recovered and assessed for IL-2 production using proliferation of the IL-2 dependent CTLL-2 cell line. Conversion of Tyr₉₇ from tyrosine to nitrotyrosine in peptide nMCC₈₈₋₁₀₃ completely abrogates recognition by 2B4.

FIG. 3 T cell specificity in mice immunized with MCC₈₈₋₁₀₃ or nMCC₈₈₋₁₀₃ peptides. CBA mice were immunized with MCC₈₈₋₁₀₃ (left panel) or nMCC₈₈₋₁₀₃ peptides (right panel) in IFA. Ten days post-immunization, draining lymph node cells were recovered and stimulated in vitro with the indicated concentrations of MCC₈₈₋₁₀₃ or nMCC₈₈₋₁₀₃ peptides. After 3 days of stimulation supernatants were recovered and assayed for the presence of secreted IFN-γ by ELISA. Data are plotted as average IFN-γ produced in replicate wells plus or minus SEM.

FIG. 4 Immune responses in PCC transgenic mice after immunizition with MCC₈₈₋₁₀₃ or nMCC₈₈₋₁₀₃ peptides. PCC transgenic mice of an H2^{k} haplotype (line 119) ¹⁹ were immunized with MCC₈₈₋₁₀₃ (upper panels:A and B) or nMCC₈₈₋₁₀₃ peptides (lower panels: C. and D)) in IFA. Ten days post-immunization, draining lymph node cells were recovered and stimulated in vitro with the indicated concentrations of MCC₈₈₋₁₀₃ or nMCC₈₈₋₁₀₃ peptides. After 3 days of stimulation cells were pulsed with tritiated thymidine to determine the level of peptide-induced proliferation.(left panels (A and C)) and supernatants were recovered and assayed for the presence of secreted IFN-γ by ELISA (right panels (B and D)). Data are plotted as the average stimulation index ([³H]-thymidine incorporation in the presence of antigen/[³H]-thymidine incorporation in the presence of medium only) of replicate wells plus the SEM (left panels) or average IFN-γ produced in replicate wells plus or minus SEM (right panels). FIGS. 4 E and F illusttate the IFNγ response in PCC transgenic mice (tolerant towards PCC/MCC) immunized subcutaneously with MCC₈₈₋₁₀₃ (E) or nitrated MCC₈₈₋₁₀₃ (F) peptides. Ten days post immunization draining lymph node cells were recovered and stimulated in vitro with varying amounts of MCC₈₈₋₁₀₃ or nitrated MCC₈₈₋₁₀₃ peptide. Responsiveness was measured in terms of IFN-γ released into the supernatant in response to antigen as measured by capture ELISA.

FIG. 5.A Responses of a panel of T cell hybridomas raised against nMCC₈₈₋₁₀₃. PCC transgenic mice of an H2^{k} haplotype (line 119)¹⁹ and wild type CBA mice were immunized with nMCC₈₈₋₁₀₃ peptide in IFA. Ten days post-immunization, splenocytes were recovered and stimulated in vitro with nMCC₈₈₋₁₀₃ peptide (10 uM) for 10 days. Activated cells were then fused with BW5147 thymoma cells to generate T cell hybridomas which were subsequently cloned by limiting dilution on 96 well plates. 5×10⁴ of each T cell hybridoma was mixed with 5×10⁴ I-E^{k}-transfected CHO cells in the presence or absence of MCC₈₈₋₁₀₃ or nMCC₈₈₋₁₀₃ peptides (10 uM). After 24 h, supernatants were recovered and assessed for IL-2 production using proliferation of the IL-2 dependent CTLL-2 cell line. Data are plotted as the average [³H]-thymidine incorporation of replicate wells of CTLL-2 cells. B. Characterization of the TCR β chain V-D-J gene usage by the nMCC₈₈₋₁₀₃-specific T cell hybridoma 119-1F5. cDNA encoding the TCR β chain was obtained by RT-PCR using degenerate TCR β chain-specific primers. Shown is the nucleotide and amino acid sequence of the β chain V-D-J junction region for the 119-1F5 hybridoma aligned with the same region from the MCC₈₈₋₁₀₃-specific T cell clone 6.9R.D6 ²⁶ Identical nucleotides are indicated as dots. The 119-1F5 sequence contains a single, positively charged arginine residue in the D region which is predicted to play a role in accommodating the negative charge acquired during conversion of tyrosine to nitrotyrosine in peptide nMCC₈₈₋₁₀₃.

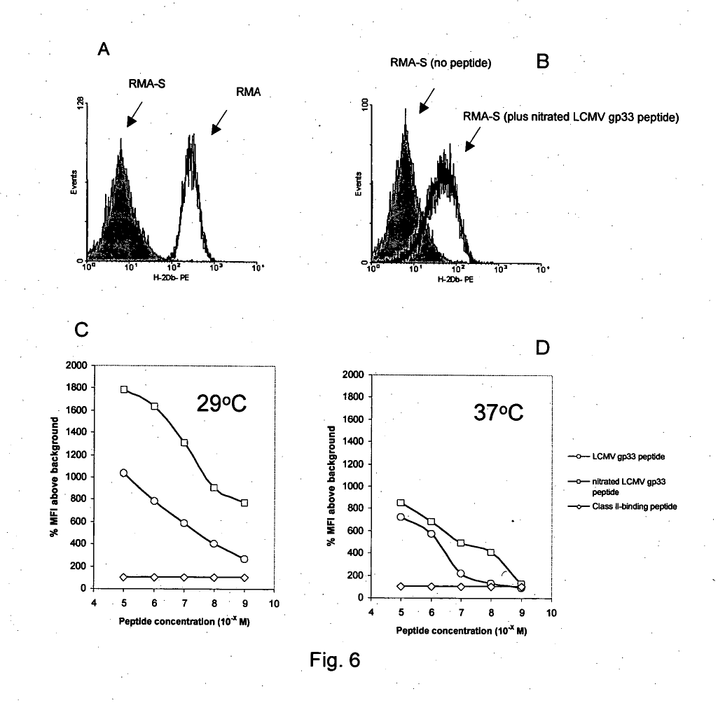

FIG. 6. A Flow cytometric analysis of MHC class I expression (H-2D^{b}) by RMA and RMA-S (TAP-deficient) cells. Cells were maintained at 37° C. and were stained with PE-conjugated anti-H-2D^{b} mAb (CTDb, Cedarlane). RMA-S cells maintained at 37° C. fail to express detectable amounts of H-2D^{b} at the cell surface. B. Surface expression of H-2D^{b} can be rescued by incubation in-the presence of nitrated LCMV gp33 peptide. RMA-S cells (maintained at 37° C.) were incubated in the presence or absence of 10 UM nitrated LCMV gp33 peptide for 4 hrs and were stained with PE-conjugated anti-H-2Db mAb (CTDb, Cedarlane). C. Rescue of H-2D^{b} surface expression using varying amounts of peptide. RMA-S cells were maintained at 29° C. (left panel) or 37° C. (right panel) for 48 hours prior to being incubated for 4 hrs at 37° C. in the presence of the indicated amounts of non-nitrated LCMV gp33 peptide, nitrated LCMV gp33 peptide or an unrelated class II-binding peptide. Cells were then stained with PE-conjugated anti-H-2D^{b} mAb (CTDb, Cedarlane). Results are reported as percentage increase in mean flourescent intensity (MFI) of cells incubated in the presence of peptide versus cells incubated in the absence of peptide. Result: both nitrated and non-nitrated peptides are capable of binding to H-2D^{b} and stabilizing its surface expression in a peptide concentration-dependent manner. Pre-incubation of cells at 29° C. allows for higher signal due to accumulation of empty of class I molecules at the cell surface prior to peptide exposure.

FIG. 7. A Flow cytometric analyses of naïve splenocytes stimulated in the presence of RMA-S cells pulsed with LCMV gp33 and nitrated LCMV gp33 peptides. RMA-S cells were maintained at 29° C. for 48 hours prior to being incubated for 4 hrs at 37° C. in the absence of peptide or in the presence of 10 uM non-nitrated LCMV gp33 peptide or 10 uM nitrated LCMV gp33 peptide. After extensive washing to remove non-bound peptides, 1×10⁶ of each RMA-S cell type (no peptide, gp33 peptide or nitrated gp33 peptide) was added to 1×10⁷ splenocytes from naïve C57BI/6 mice in a single well of a 6-well plate in a total volume of 2 ml culture media. After 4 days of culture IL-2 was added (10 U/ml). After a further 3 days of culture cells were recovered and analyzed by flow cytometry to detect expression of the cell surface markers CD4 and CD8.

FIG. 8. Flow cytometric analyses of TCR V beta repertoire usage by naïve splenocytes stimulated in the presence of RMA-S cells pulsed with LCMV gp33 and nitrated LCMV gp33 peptides. Splenocytes from naïve C57BI/6 mice were incubated with peptide-pulsed RMA-S cells as described in FIG. 7 and 7 days later the TCR V beta gene usage was characterized by flow cytometry. Results are shown for V beta 8.3 and V beta 9, the two subtypes that are most common after stimulation with RMA-S cells pulsed with LCMV gp33 and nitrated LCMV gp33.

FIG. 9. Peptide-specific cytolytic activity of T cells that are expanded after incubation of naïve splenocytes in the presence of RMA-S cells pulsed with nitrated LCMV gp33 peptides. Splenocytes from naïve C57BI/6 mice were incubated with nitrated LCMV gp33 peptide-pulsed RMA-S cells as described in FIG. 7 and 8 days later were analyzed for their ability to kill EL4 tumor targets pulsed with LCMV gp33 and nitrated LCMV gp33 peptides in a standard cytotoxicity assay. Splenocytes activated in the presence of nitrated LCMV gp33 kill tumor targets pulsed with nitrated gp33 at a higher rate than tumor targets pulsed with non-nitrated peptide.

What is claimed is:

1. A T cell line specific for a nitrotyrosine-containing compound.

2. The T cell line of claim 1 wherein the T cell line is a T helper (CD 4+) or T cytotoxic (CD 8+) cell line.

3. The T cell line of claim 2 wherein the T cell line is a cytotoxic T cell line of claim 1, wherein the cell line is specific to a nitrotyrosine-containing compound.

4. The T cell line of claim 2, wherein the nitrotyrosine containing compound is a self-antigen comprising a tyrosine to nitrotyrosine conversion or an epitope of the self-antigen comprising the conversion.

5. An isolated solubilized T cell receptor isolated from the T cell of claim 1 specific for a nitrotyrosine-containing compound.

6. The isolated solubilized T cell receptor of claim 5 comprising the amino acid SEQ. ID. NO. 4.

7. An antibody specific for a self-antigen comprising a tyrosine to nitrotyrosine conversion.

8. The antibody of claim 7 wherein the antibody is a monoclonal antibody.

9. The antibody of claim 7 wherein the self-antigen is a class 1 or class II MHC or epitope thereof that has said conversion.

10. A hybridoma cell line that produces the antibody of claim 8.

11. The antibody of claim 8 wherein said antibody is a humanized antibody.

12. A pharmaceutical composition comprising the antibody of claim 11 and a pharmaceutically acceptable carrier.

13. A self-antigen comprising a tyrosine to nitrotyrosine conversion and that induces a specific immune response to said converted self-antigen.

14. The self-antigen of claim 14 wherein said immune response is a cellular immune response.

15. A pharmaceutical composition comprising the self-antigen of claim 13 and a pharmaceutically acceptable carrier.

16. A method of inducing a cellular immune response specific to a nitrotyrosine-containing compound comprising administering an effective amount of the nitrotyrosine containing compound.

17. The method of claim 16, wherein the nitrotyrosine-containing compound is a self-antigen having a tyrosine to nitrotyrosine conversion.

18. The method of claim 17 wherein the self-antigen is a tumour specific antigen having a tyrosine to nitrotyrosine conversion.

19. The method of claim 16 wherein cytotoxic T cells specific to the nitrotyrosine-containing compound are generated.

20. A method of inhibiting a cellular immune response specific to a nitrotyrosine-containing compound comprising administering an effective amount of an anti-nitrotyrosine-containing compound antibody

21. The method of claim 20 wherein the nitrotyrosine-containing compound is a self-antigen having a tyrosine to nitrotyrosine conversion.

22. A method of identifying a modulator of a nitrotyrosine-containing ligand induced cellular immune response comprising:
(a) incubating an effective amount of nitrotyrosine-containing ligand and T-cells specific to said nitrotyrosine-containing ligand under conditions that promote ligand:T cell complex formation in the presence of a potential modulator;
(b) assaying for one or more of the following to determine the effect of the potential modulator on nitrotyrosine-containing ligand:T-cell complex formation:
(i) unbound nitrotyrosine-containing ligand;
(ii) unbound T-cells;
(iii) unbound potential modulator;
(iv) bound nitrotyrosine-containing ligand to T-cells;
(v) bound nitrotyrosine-containing ligand to the potential modulator;
(vi) bound T-cells to the potential modulator;
(vii) activation of T-cells bearing nitrotyrosine-specific receptors as determined by proliferation and/or cytokine production and/or expression of ligand specific T cell receptors;
(c) comparing the assayed level with that of a control wherein any change from a control level is indicative that the potential modulator is a modulator.

23. The method of claim 22 wherein the nitrotyrosine-containing ligand is a self-antigen having a tyrosine to nitrotyrosine conversion.

24. The method of claim 23 wherein the self-antigen is a tumour specific antigen.

25. The method of claim 21 wherein the modulator is an inhibitor of the immune response wherein any reduction in binding of nitrotyrosine-containing ligand and T-cell as compared to control or higher levels of unbound nitrotyrosine-containing ligand or T-cells as compared to a control or lower levels of free modulator or higher levels of bound modulator and ligand or modulator and T cell; or lower level of activated T cells as compared to a control would be indicative of an inhibitor

26. The method of claim 25 wherein the inhibitor comprises an antibody against the nitrotyrosine-containing compound or the nitrotyrosine-containing compound specific T cell receptor of the T cell.

27. The method of claim 21 wherein the modulator is an inducer of the immune response wherein any increase in binding of nitrotyrosine-containing ligand and T-cell as compared to control or lower levels of unbound nitrotyrosine-containing ligand or T-cells as compared to a control or higher levels of free modulator or lower levels of bound modulator and ligand or modulator and T cell; or higher level of activated T cells as compared to a control would be indicative of an inducer

28. The method of claim 23 for identifying self-antigens having a tyrosine to nitrotyrosine conversion that are antigenic comprising: administering the converted self-antigen to an animal and detecting CD8+ and/or antibody production or antigen/cell or antigen/antibody complex formation, wherein said production or complex formation is indicative of an antigenic converted self-antigen.

29. A method of identifying a medical condition that is associated with a nitrotyrosine-containing compound by:
(a) obtaining a sample from the patient that is affected by said condition;
(b) screening the sample for one or more of the following:
(i) nitrotyrosine-containing compounds;
(ii) nitrotyrosine-containing compounds in complex with T cells; or
(iii) nitrotyrosine-containing compound specific T cells;
(c) identifying said nitrotyrosine-containing compound;
(d) comparing the results of b and c with a control sample obtained from condition free tissue,
wherein the presence of nitrotyrosine containing compounds or specific T cells or complex formation is indicative of a medical condition, associated with a nitrotyrosine containing compound.

30. The method of claim 29 wherein nitrotyrosine is present in the sample and the condition is an autoimmune disease selected from the group of autoimmune diseases consisting of: multiple sclerosis, inflammatory bowel disease, celiac disease, arthritis, autoimmune diabetes, autoimmune uveitis, allergic encephalomyelitis, or systemic lupus erythematosus.

31. The method of claim 29 wherein nitrotyrosine is present in the sample and is indicative of a disease with an inflammatory component such as transplant rejection, Alzheimer's disease, ischemia reperfusion injury, pneumonia, leishmaniasis.

32. A method for treating a medical condition associated with a nitrotyrosine-containing compound induced cellular immune response comprising administering to a patient in need thereof a modulator of said immune response.

33. The method of claim 32 wherein the nitrotyrosine-containing compound is a self-antigen.

34. The method of claim 33 wherein the medical condition is an inflammatory or autoimmune disease.

35. The method of claim 34 wherein modulator is an inhibitor of the nitrotyrosine-induced cellular immune response.

36. The method of claim 35 wherein the inhibitor is an antibody to said nitrotyrosine-containing compound or T cell specific receptor to said compound.

37. A method of preventing a medical condition associated with a nitrotyrosine-containing compound comprising immunizing a patient with said nitrotyrosine-containing compound.

38. The method of claim 33 wherein the medical condition is cancer and the self-antigen a tumor-associated or tumor-specific peptide or protein containing a tyrosine to nitrotyrosine conversion together with a pharmaceutically acceptable carrier which induces a specific immune response to said tumor.

39. The method of claim 33 wherein the medical condition is an infectious disease caused by a cell or microbe infection and the modulator is a cellular or microbial peptide or a cellular or microbial protein containing a tyrosine to nitrotyrosine conversion that induces an antibody and/or cellular immune response against cells that are infected with the microbe.

40. The method of claim 34 wherein the medical condition is an autoimmune disease that is associated with a self-antigen comprising a tyrosine to nitrotyrosine conversion and the modulator is an anti-nitrotyrosine self-antigen specific monoclonal antibody.


SEQUENCE LISTING (7 sequences)

SEQ ID NO: 1 - protein, 17 aa, Artificial Sequence
  Pigeon Cytochrome C (PCC 88-104)
KAERADLIAYLKQATAK

SEQ ID NO: 2 - protein, 16 aa, Artificial Sequence
  Moth Cytochrome C (MCC 88-103)
ANERADLIAYLKQATK

SEQ ID NO: 3 - protein, 16 aa, Artificial Sequence
  Moth Cytochrome C (nMCC 88-103) with nitrotyrosine in place of tyrosine at amino acid number 10
ANERADLIAXLKQATK

SEQ ID NO: 4 - protein, 18 aa, Artificial Sequence
  VD5 region of T Cell Receptor Beta Chain (TCR B) of Mouse nMCC 88-103 specific TCR (119-1F5)
SAVYFCASSRDNSNSDYT

SEQ ID NO: 5 - DNA, 54 bp, Artificial Sequence
  Nucleic acid coding region of VDJ region of T Cell Receptor Beta Chain (TCR B) of Mouse MCC 88-103 specific TCR (119-1F5)
tcagctgtctatttttgtgccagcagccgggacaattcaaactccgactacacc

SEQ ID NO: 6 - protein, 17 aa, Artificial Sequence
  VDJ region of T Cell Receptor Beta Chain (TCR B) of Mouse MCC (88-103) specific TCR (69R.D6)
SAVYFCASSQDQNSDYT

SEQ ID NO: 7 - DNA, 51 bp, Artificial Sequence
  Nucleic acid coding region of VDJ region of T Cell Receptor Beta chain (TCR B)  of Mouse (MCC 88-103) specific TCR (69R.D6)
tcagctgtctatttttgtgccagcagccaagatcagaactccgactacacc